(19) United States
(12) Patent Application Publication
(10) Pub. No.: US 2024/0050751 A1
(43) Pub. Date: Feb. 15, 2024

(54) CHRONIC CARDIAC DYSFUNCTION PREVENTION

(71) Applicant: Presidio Medical, Inc., South San Francisco, CA (US)

(72) Inventors: Thomas Flores, San Francisco, CA (US); Andrew Sullivan, Boulder, CO (US)

(21) Appl. No.: 18/300,335

(22) Filed: Apr. 13, 2023

Related U.S. Application Data
(60) Provisional application No. 63/331,157, filed on Apr. 14, 2022.

(51) Int. Cl. A61N 1/36

(52) U.S. Cl. CPC A61N 1/36114; A61N 1/36062; A61N 1/36157

(57) ABSTRACT

A method to prevent formation of unfavorable sequelae includes initiating delivering a neural inhibiting therapy to a nerve associated with an extracardiac sympathetic nervous system of a patient within 40 days of an onset of symptoms associated with acute coronary syndrome. The neural inhibiting therapy includes delivering an electrical therapeutic signal to the nerve sufficient to substantially prevent neural activity of the nerve from occurring. The method also includes continuing delivery of the neural inhibiting therapy to the nerve associated with the extracardiac sympathetic nervous system for a desired treatment period and discontinuing delivery of the neural inhibiting therapy to the nerve after the desired treatment period and enabling neural activity of the nerve to resume.

### CROSS-REFERENCE TO RELATED APPLICATIONS

The present application claims priority from U.S. Provisional No. 63/331,157, filed Apr. 4, 2022, which is incorporated by reference in its entirety.

### TECHNICAL FIELD

This application relates, in some embodiments, to facilitating inhibition of biological signals through nerve tissue.

### BACKGROUND

The gate control theory of pain was developed in the 1960s and led to the advent of stimulation-based pain management therapies to reduce pain inputs from reaching the brain by selectively stimulating non-nociceptive fibers (non-pain transmitting fibers) in the spinal cord to inhibit transmission of pain stimuli to the brain. Current stimulation systems typically utilize electrical stimulation signals in the <100 Hz frequency range, and recently in the kHz frequency range. Some newer stimulation systems provide stimulations signals in the <1 Hz frequency range. In addition, since the 1960s, many additional uses for stimulation-based therapy have been developed.

### SUMMARY

Acute coronary syndrome (ACS) are life-altering events that severely impact cardiac health in both the acute and chronic settings. Cardiac ischemia, as a result of reduced perfusion through the coronary arteries, leads to the formation of an infarct, a region of dead and non-conductive cardiac tissue. The resulting tissue death leads to degeneration of cardiac function, importantly via increased myocyte stiffness, which impedes adequate contraction, and generation of regional sites of varying conductivity, which leads to proarrhythmic substrates. The extracardiac circuit is also affected through neural remodeling of the autonomic system, including at the stellate ganglion. The combination of intra and extracardiac remodeling can leave a patient at risk for heart failure and lethal cardiac arrhythmias, both of which may be accelerated by a positive feedback loop of sympathetic overactivity.

AMIs are classified into two major groups depending on their electrocardiographic fingerprint, extent of cardiac death, and need for response: ST elevated myocardial infarctions (STEMI) and non-ST elevated myocardial infarctions (NSTEMI). While both STEMIs and NSTEMIs require immediate attention, STEMIs require immediate intervention to prevent the sudden death and/or formation of a catastrophic infarct. Patients with NSTEMIs are carefully monitored over 24-72 hours and may receive interventional treatment if the prognosis looks unfavorable.

The post-infarct patient timeline (see FIG. 1) consists of a series of high-risk complications, including ventricular arrhythmias (VAs), bradyarrhythmias, cardiogenic shock, stroke, ischemic mitral regurgitation, papillary muscle rupture, ventricular septal rupture, LV free wall rupture, and pericarditis, in order from hyperacute to subacute onset. Further, atrial fibrillation may occur at any time in the post-infarct setting. The incidence of sudden cardiac death (SCD) as a result of ventricular arrhythmias can be segmented into 3 major timeframes: hyperacute (generally <48 hours), subacute/acute (generally 48 hours—40 days) and chronic (generally >40-90 days). The hyperacute arrhythmias tend to be a direct result of the ischemic tissue, while the subacute and chronic arrhythmias result from cardiac remodeling. Improvements in cardiac perfusion, namely time to reperfusion, have significantly reduced the incidence of SCD during the hyperacute timeframe. However, ventricular arrhythmias during the subacute and chronic timeframes still present a problem, with sudden deaths resulting from a presumed arrhythmia.

There is a significant increase in ventricular arrhythmias in the chronic phase compared to the subacute phase. This may be the result of neural remodeling in the cardiac substrate and autonomic nerves that occurs during the subacute phase despite adequate reperfusion during the hyperacute timeframe. These remodeled substrates are both intrinsically arrhythmogenic and hypersensitive to sympathetic tone, leading to ventricular arrhythmias. Reduction in infarct size and proarrhythmic remodeling prior to the chronic phase can reduce arrhythmia incidence in patients compared to those with large infarcts and highly remodeled tissue.

Pharmaceutical therapies are used to reduce the progression of infarct size and incidence of SCD. However, adequate titration of these medications during the subacute phase is difficult, resulting in inadequate therapy. If an infarct size is very large, it can give rise to heart failure with reduced ejection fraction (HFrEF) or a proarrhythmic substrate. At this stage, an implantable cardioverter defibrillator (ICD) is sometimes used as primary or secondary prevention of SCD. While ICDs can be effective at preventing SCD by cardioverting patients during an arrhythmic event, they do not help to treat the underlying disease state. Further, these devices are not recommended to be implanted until the chronic timeframe, after neural remodeling has already occurred. These guidelines are the result of studies that compared survival of ICDs deployed during the subacute and chronic timeframes; because the relative cause of SCD due to cardiac arrhythmias in the subacute phase is less than that of non-arrhythmic events (e.g., cardiogenic shock, etc.), improved survival was not demonstrated. Nevertheless, arrhythmic events still occur during this timeframe, which has led to the development of less invasive defibrillator devices such as wearable ICDs that can be justified due to their lack of surgery and comparatively reduced cost. However, these devices do nothing to slow the progression of the remodeling that puts the patients at risk for long-term mortality.

There is therefore a need for a way to prevent the formation of a chronic proarrhythmic cardiac and stellate substrate in patients hospitalized for acute myocardial infarction in order to reduce unfavorable cardiac outcomes, including reducing infarct size, improving electrical stability, improving cardiac output, and reducing progression of heart failure, during the subacute post-MI timeframe. There is also a need to treat the already formed infarct in the chronic post-MI timeframe to suppress cardiac arrhythmias and enable reverse remodeling of the cardiac and stellate substrate. Further, an ideal solution should also help prevent the incidence of ventricular arrhythmias that lead to sudden cardiac death. Improvement in non-VA mortality during the subacute timeframe would also be desirable.

In some aspects, the techniques described herein relate to a method to prevent formation of chronic proarrhythmic cardiac and stellate substrates, including: initiating delivering of a neural inhibiting therapy to a nerve associated with an extracardiac sympathetic nervous system of a patient within 40 days of an onset of symptoms associated with an acute myocardial infarction (MI) event, wherein the neural inhibiting therapy includes delivering an electrical therapeutic signal to the nerve sufficient to substantially prevent neural activity of the nerve from occurring; continuing delivery of the neural inhibiting therapy to the nerve associated with the extracardiac sympathetic nervous system for a desired treatment period; and discontinuing delivery of the neural inhibiting therapy to the nerve after the desired treatment period and enabling neural activity of the nerve to resume.

In some aspects, the techniques described herein relate to a method, further including implanting an electrical contact close to the nerve associated with the extracardiac sympathetic nervous system of the patient.

In some aspects, the techniques described herein relate to a method, wherein implanting includes implanting the electrical contact under ultrasound or fluoroscopy or other image guidance method.

In some aspects, the techniques described herein relate to a method, wherein delivering therapy to the nerve associated with the extracardiac sympathetic nervous system of the patient includes delivering therapy to a paravertebral chain at thoracic levels T1-T2, stellate ganglia, spinal cord, ansa subclavia or dorsal root ganglia.

In some aspects, the techniques described herein relate to a method, wherein delivering therapy occurs within 14 days of the MI event, within 2 days of the MI event, before 14 days of the MI event, or during the MI event.

In some aspects, the techniques described herein relate to a method, wherein delivering therapy includes one or more of delivering a neural inhibiting electrical waveform, delivering an ultra-low frequency waveform, providing a non-electrical therapy, providing thermal energy, providing magnetic energy, or providing ultrasonic energy.

In some aspects, the techniques described herein relate to a method, wherein continuing neural inhibiting including inhibiting neural activity for at least 14 days, up to 40 days, until a sympathetic tone is decreased by a desired amount in the absence of the neural inhibiting, until a biomarker level of a biomarker relating to myocardial death drops by a desired amount.

In some aspects, the techniques described herein relate to a method, wherein the biomarker is troponin or any other biomarker described herein.

In some aspects, the techniques described herein relate to a method, wherein deactivating blocking includes deactivating a waveform generator.

In some aspects, the techniques described herein relate to a method, further including removing a treatment device from the patient, wherein the treatment device is configured to perform the method.

In some aspects, the techniques described herein relate to a method, wherein removing includes removing an implant or removing a patch or other device from a skin surface of a patient.

In some aspects, the techniques described herein relate to a system configured to prevent formation 1 through 11.

In some aspects, the techniques described herein relate to a method to reduce a cardiotoxic sympathetic tone in an extracardiac neural circuit, including: initiating delivering of a neural inhibiting therapy to a nerve associated with an extracardiac sympathetic nervous system of a patient within 40 days of an onset of symptoms associated with an acute myocardial infarction (MI) event, wherein the neural inhibiting therapy includes delivering an electrical therapeutic signal to the nerve sufficient to substantially prevent neural activity of the nerve from occurring; continuing delivery of the neural inhibiting therapy to the nerve associated with the extracardiac sympathetic nervous system for a desired treatment period; and discontinuing delivery of the neural inhibiting therapy to the nerve after the desired treatment period and enabling neural activity of the nerve to resume.

In some aspects, the techniques described herein relate to a method, further including implanting an electrical contact close to the nerve associated with the extracardiac sympathetic nervous system of the patient.

In some aspects, the techniques described herein relate to a method, wherein implanting includes implanting the electrical contact under ultrasound or fluoroscopy or other image guidance method.

In some aspects, the techniques described herein relate to a method, wherein delivering therapy to the nerve associated with the extracardiac sympathetic nervous system of the patient includes delivering therapy to a paravertebral chain at thoracic levels T1-T2, stellate ganglia, spinal cord, ansa subclavia or dorsal root ganglia.

In some aspects, the techniques described herein relate to a method, wherein delivering therapy occurs within 14 days of the MI event, within 2 days of the MI event, before 14 days of the MI event, or during the MI event.

In some aspects, the techniques described herein relate to a method, wherein delivering therapy includes one or more of delivering a neural inhibiting electrical waveform, delivering an ultra-low frequency waveform, providing a non-electrical therapy, providing thermal energy, providing magnetic energy, or providing ultrasonic energy.

In some aspects, the techniques described herein relate to a method, wherein continuing neural inhibiting including inhibiting neural activity for at least 14 days, up to 40 days, until a sympathetic tone is decreased by a desired amount in the absence of the neural inhibiting, until a biomarker level of a biomarker relating to myocardial death drops by a desired amount.

In some aspects, the techniques described herein relate to a method, wherein the biomarker is troponin or any other biomarker described herein.

In some aspects, the techniques described herein relate to a method, wherein deactivating neural inhibiting includes deactivating a waveform generator.

In some aspects, the techniques described herein relate to a method, further including removing a treatment device from the patient, wherein the treatment device is configured to perform the method.

In some aspects, the techniques described herein relate to a method, wherein removing includes removing an implant or removing a patch or other device from a skin surface of a patient.

In some aspects, the techniques described herein relate to a system configured to prevent formation 13 through 23.

In some aspects, the techniques described herein relate to a method to reverse formation of chronic proarrhythmic cardiac and stellate substrates, including: initiating delivering of a neural inhibiting therapy to a nerve associated with an extracardiac sympathetic nervous system of a patient 40 days or more after occurrence of a symptom associated with an acute myocardial infarction (MI) event, wherein the neural inhibiting therapy includes delivering an electrical therapeutic signal to the nerve sufficient to substantially prevent neural activity of the nerve from occurring; continuing delivery of the neural inhibiting therapy to the nerve associated with the extracardiac sympathetic nervous system; assessing a function of cardiac tissue that is different from infarcted cardiac tissue; and discontinuing delivery of the neural inhibiting therapy to the nerve and enabling neural activity of the nerve to resume in response the assessing.

In some aspects, the techniques described herein relate to a method, further including implanting an electrical contact close to the nerve associated with the extracardiac sympathetic nervous system of the patient.

In some aspects, the techniques described herein relate to a method, wherein implanting includes implanting the electrical contact under ultrasound or fluoroscopy or other image guidance method.

In some aspects, the techniques described herein relate to a method, wherein delivering therapy to the nerve associated with the extracardiac sympathetic nervous system of the patient includes delivering therapy to a paravertebral chain at thoracic levels T1-T2, stellate ganglia, spinal cord, ansa subclavia or dorsal root ganglia.

In some aspects, the techniques described herein relate to a method, wherein delivering therapy includes one or more of delivering a neural inhibiting electrical waveform, delivering an ultra-low frequency waveform, providing a non-electrical therapy, providing thermal energy, providing magnetic energy, or providing ultrasonic energy.

In some aspects, the techniques described herein relate to a method, wherein continuing neural inhibiting including inhibiting neural activity for at least 14 days, up to 40 days, until a sympathetic tone is decreased by a desired amount in the absence of the neural inhibiting, until a biomarker level of a biomarker relating to myocardial death drops by a desired amount.

In some aspects, the techniques described herein relate to a method, wherein the biomarker is troponin or any other biomarker described herein.

In some aspects, the techniques described herein relate to a method, wherein deactivating neural inhibiting includes deactivating a waveform generator.

In some aspects, the techniques described herein relate to a method, further including removing a treatment device from the patient, wherein the treatment device is configured to perform the method.

In some aspects, the techniques described herein relate to a method, wherein removing includes removing an implant or removing a patch or other device from a skin surface of a patient.

In some aspects, the techniques described herein relate to a system configured to prevent formation 25 through 34.

In some aspects, the techniques described herein relate to a method to reduce a cardiotoxic sympathetic tone in an extracardiac neural circuit, including: initiating delivering of a neural inhibiting therapy to a nerve associated with an extracardiac sympathetic nervous system of a patient 40 days or more after occurrence of a symptom associated with an acute myocardial infarction (MI) event, wherein the neural inhibiting therapy includes delivering an electrical therapeutic signal to the nerve sufficient to substantially prevent neural activity of the nerve from occurring; continuing delivery of the neural inhibiting therapy to the nerve associated with the extracardiac sympathetic nervous system for a desired treatment period; assessing a function of cardiac tissue that is different from infarcted cardiac tissue; and discontinuing delivery of the neural inhibiting therapy to the nerve after the desired treatment period and enabling neural activity of the nerve to resume in response to the assessing.

In some aspects, the techniques described herein relate to a method, further including implanting an electrical contact close to the nerve associated with the extracardiac sympathetic nervous system of the patient.

In some aspects, the techniques described herein relate to a method, wherein implanting includes implanting the electrical contact under ultrasound or fluoroscopy or other image guidance method.

In some aspects, the techniques described herein relate to a method, wherein delivering therapy to the nerve associated with the extracardiac sympathetic nervous system of the patient includes delivering therapy to a paravertebral chain at thoracic levels T1-T2, stellate ganglia, spinal cord, ansa subclavia or dorsal root ganglia.

In some aspects, the techniques described herein relate to a method, wherein delivering therapy includes one or more of delivering a neural inhibiting electrical waveform, delivering an ultra-low frequency waveform, providing a non-electrical therapy, providing thermal energy, providing magnetic energy, or providing ultrasonic energy.

In some aspects, the techniques described herein relate to a method, wherein continuing neural inhibiting including inhibiting neural activity for at least 14 days, up to 40 days, until a sympathetic tone is decreased by a desired amount in the absence of the neural inhibiting, until a biomarker level of a biomarker relating to myocardial death drops by a desired amount.

In some aspects, the techniques described herein relate to a method, wherein the biomarker is troponin or any other biomarker described herein.

In some aspects, the techniques described herein relate to a method, wherein deactivating blocking includes deactivating a waveform generator.

In some aspects, the techniques described herein relate to a method, further including removing a treatment device from the patient, wherein the treatment device is configured to perform the method.

In some aspects, the techniques described herein relate to a method, wherein removing includes removing an implant or removing a patch or other device from a skin surface of a patient.

In some aspects, the techniques described herein relate to a system configured to prevent formation 36 through 45.

### BRIEF DESCRIPTION OF THE DRAWINGS

FIG. 1 illustrates a post-myocardial infarction timeline with and without cardiac neural inhibiting therapy.

FIG. 2 illustrates a post-injury timeline with and without neural inhibiting therapy.

FIG. 3 is a flowchart illustrating one method of preventing adverse healing after an injury, such as preventing formation of chronic proarrhythmic cardiac and stellate substrates after a myocardial infarction.

FIG. 4 is a flowchart illustrating another method of preventing adverse healing after an injury, such as preventing formation of chronic proarrhythmic cardiac and stellate substrates after a myocardial infarction.

FIG. 5A shows an embodiment of an electron-ion conversion current cell (EICCC) electrode in which an electrode is immersed in an electrolyte solution which is in contact with an ion-conductive material-electrolyte solution interface with an ion-conductive material that electrically contacts the nerve tissue N or area proximal to the nerve tissue N.

FIG. 5B is a graph illustrating by pushing a constant current from the current source, the mass of the AgCl electrode can decrease during a cathodic current (reduction reaction) and then increase with an anodic current during an oxidative reaction.

FIG. 5C shows how current being delivered to the electrode cell (EICCC) (top) can be associated with charge delivered to the electrode cell-nerve interface (middle) to provide nerve inhibition (including nerve block) (bottom) while enabling zero net charge transfer with long charge phases, according to some embodiments of the invention.

FIG. 5D illustrates waveform patterns delivered to an electrode cell to facilitate nerve inhibition (including nerve block).

FIG. 5E shows an embodiment of an EICCC connected via an electrically insulated lead to a current source.

FIG. 5F illustrates a configuration when the electron current is of one polarity as designated by the positive axis, the nerve block (or inhibition) is shown to be active and when the polarity of the current is reversed as designated by the negative axis, the nerve block (or inhibition) is shown to be inactive.

FIG. 5G shows a similar configuration to FIG. 5E but with sequestration screens that respectively separate the traditional electrode from the ion conductor and the ion conductor from the nerve itself.

FIG. 5H illustrates current vs. time and nerve block status vs. time charts similar to FIG. 5F.

FIG. 5I shows a similar configuration to FIG. 5H but also includes a feedback sensor that monitors the state of the nerve tissue and/or region proximal to the nerve N.

FIG. 5J illustrates current vs. time and nerve block status vs. time charts similar to FIG. 1I.

FIG. 6A shows a dual electrode system in which two EICCCs interface with a nerve, according to some embodiments.

FIG. 6B shows two electrodes driven with currents of opposite polarities as a function of time such that when one is in an active blocking phase, the other is in an inactive non-blocking phase which resets the electrode for blocking once the current polarity is again reversed.

FIGS. 7A-7B show an embodiment where dual traditional electrodes interface with a nerve but are driven from a current source via electrically insulated leads with currents of opposite polarities such that when one is in a blocking phase, the other is in a non-blocking phase which resets the electrode for blocking once the current polarity is again reversed.

FIGS. 7C-7D show an embodiment where dual EICCCs interface with a nerve N but are driven with currents of opposite polarities such that when one is in a blocking phase, the other is in a non-blocking phase which resets the electrode for blocking once the current polarity is again reversed.

FIG. 8A shows an embodiment of an EICCC electrode in which an electrode is immersed in an electrolyte solution which fluidly in is contact with an ion-conductive material such as a hydrogel, gel or other polymer that electrically contacts the nerve tissue or area proximal to the nerve tissue.

FIG. 8B shows a system similar to that shown in FIG. 8A with the addition of a reference electrode in proximity to the electrode (working electrode) to monitor voltage drop across the working electrode for EICCC monitoring purposes.

FIG. 8C shows a system similar to that shown in FIG. 8A with the addition of a reference electrode in proximity to the nerve tissue interface to monitor voltage drop across the EICCC to the nerve tissue for EICCC monitoring purposes.

FIGS. 8D-8F show an embodiment of an electrode lead configured to plug into and extend from a current source (not shown, near end) that might take the form of conventional IPGs (implantable pulse generators).

FIGS. 9A-C illustrate an embodiment of an EICCC electrode in which multiple tissue interfaces are present on the electrode and are individually addressable.

FIG. 10 shows a schematic embodiment of an EICCC integrated within a hermitically sealed enclosure which contains the current source, battery or power supply, and controller to drive the EICCC.

FIG. 11A shows an embodiment of a system having an electrode configuration in which two electrode contacts are housed within the same electrically insulated enclosure.

FIG. 11B shows one embodiment of current waveforms delivered by the two electrodes of the system of FIG. 11A.

FIG. 12A illustrates one embodiment of a neuromodulation waveform suitable for neuromodulation inhibition according to the therapeutic methods described herein.

FIG. 12B illustrates three additional embodiment of neuromodulation waveforms suitable for neuromodulation inhibition according to the therapeutic methods described herein.

FIG. 12C is table describing parameters of various neuromodulation waveforms suitable for neuromodulation inhibition according to the therapeutic methods described herein.

FIG. 13 illustrates graphs showing the current at three electrodes during operation of a neuromodulation inhibition system in bipolar mode and providing a bias (or offset) current.

FIGS. 14A-14C schematically illustrate a generally tubular (e.g., cylindrical) lead contact that can include a sidewall and an interior lumen.

FIGS. 15A-15B schematically illustration additional lead embodiments, including conduits and spaced-apart lead contacts, such as those illustrated in FIGS. 14A-14C.

FIG. 16 illustrates sympathetic ganglia, including the cervical and stellate (cervicothoracic) ganglia, along with their innervation targets in the heart.

### DETAILED DESCRIPTION

This application describes, in some aspects, methods and systems for providing a reversible neural inhibiting therapy within a predetermined time period from the occurrence of an adverse physiological event or injury. The reversible neural inhibiting therapy blocks, suppresses, reduces, or inhibits the activity of one or more nerves, thereby enable the body to naturally recover, heal, remodel, etc., without the influence of the otherwise active nerves.

In one example, the reversible neural inhibiting therapy inhibits cardiac nerve activity to reduce the overall sympathetic tone post myocardial infarction. As discussed further below, reducing sympathetic tone enables non-harmful cardiac remodeling, and therefore, reduced risk of future ventricular arrythmia and sudden cardiac death. Different nerves may be reversibly inhibited after other adverse physiological events occur to enable similar non-harmful, and even improve recovery, as discussed in further detail, below.

Reversible neural inhibiting can include application of direct current (DC), low frequency direct current, high frequency current, or non-electrical therapy (e.g., drug therapy, etc.) to facilitate nerve inhibition, including nerve block, nerve suppression, nerve hypersuppression, or nerve block without rapid reversibility or recovery after therapy has been removed or stopped.

In one embodiment, an electrical stimulator interfaces with the target nerve via ionic conduction pathways instead of conventional electrodes that that do not have an ionic conduction component. Such technology may provide an additional advantage of enabling intermittent or continuous short-term and long-term nerve block can while reducing risk of damage to the nerve cells.

Although any of a variety of pulse generators, leads, electrodes, and electrical waveforms may be used to achieve the desired neural inhibition, in some embodiments, systems and electrodes for safely delivering inhibiting or blocking direct current (DC) to neural tissue deliver cycled cathodic and anodic current through a high-charge chemistry. Tissue safety can be maintained by separating the metal interface from the nerve tissue with an ionically conductive element, and by operating the electrode below reaction potentials for undesired reactions, such as electrolysis of water, or oxidation and reduction of water (H2O), which create harmful reactive species such as OH−, H+ or oxygen free radicals.

In other aspects, systems and methods for inhibiting the neural activity of the cardiac nervous system include methods of delivering such nerve inhibiting therapy to a patient and targeting particular nerves of interest. Indeed, the term “inhibiting,” as used herein shall refer to affecting the function of a targeted nerve by inhibiting, partially inhibiting, blocking, partially blocking, suppressing, partially suppressing, reducing, partially reducing, or otherwise diminishing the performance of the targeted nerve.

As mentioned above, increased sympathetic tone in patients after an acute myocardial infarction can be directly responsible for development of infarcts with high complications. Medications and procedures aim to reduce the sympathetic tone to stifle the progression of this infarct, but also can be used to result in reverse remodeling of the heart to “heal” the infarct. One difficulty with existing therapies is they cannot adequately suppress the sympathetic tone without systemic complications (e.g., oral medications, etc.), without constant care (e.g., daily lidocaine injections, etc.), putting the patient at risk (e.g., continuous infusion of lidocaine using an outpatient pump, etc.), or iatrogenic, undesirable side effects (e.g., long-lasting Horner's syndrome from ablative/surgical techniques, etc.).

The sympathetic tone can be modulated via several nerve sites within the body, including the thoracic spinal cord and the thoracic paravertebral sympathetic chain. Notably, the paravertebral sympathetic chain at the thoracic levels T1-T2 serves as the major conduit for sympathetic control of the heart. Targeting this region with a way to reduce electrical activity results in reduced sympathetic tone.

In one embodiment, a method of reducing the sympathetic tone to impede progression of an infarct includes delivering an electrical waveform capable of reducing or blocking nerve activity to the paravertebral sympathetic chain (or any other therapy described below for reducing or blocking nerve activity to the paravertebral sympathetic chain) shortly after the onset of an acute MI event. A therapeutic device (such as an electrical signal generator, including, but not limited to, any of the electrical nerve inhibition signal generators, leads, electrodes, waveforms and systems described below) can be placed under ultrasound guidance at the bedside or under fluoroscopic guidance during reperfusion. Delivery of the inhibiting waveform will decrease nerve activity, including sympathetic tone to the heart, during at least the duration of the subacute phase (at least ˜14 days) to prevent the formation of the proarrhythmic cardiac substrate and paravertebral chain. The therapy may be turned on while the patient is still in the hospital and continue with the patient after discharge. Once the patient has exited the window during which remodeling is expected to occur, the device can be removed and/or decoupled from the patient. The explicit disease setting, physiologic outcome, and usage for and required materials to enable such a system are novel. The ability to deliver such a therapy in a minimally invasive (e.g., percutaneous, etc.) approach is advantageous for the success of the therapy, as well.

Electrode leads may be placed close to the paravertebral chain at thoracic levels T1-T2. This is slightly caudal to the typical location for stellate ganglion blocks. The electrodes are designed such that they can deliver the electronic (e.g., ultra-low frequency or ULF, or any other) waveform safely with zero or minimal irreversible effects, and the leads are designed to prevent migration away from the target region while also enabling non-invasive explanation.

In another embodiment, therapy is provided after an infarct has already formed. Reduction in sympathetic tone may help with reverse remodeling of the infarct tissue, to reduce chronic morbidity/mortality.

Acute coronary syndromes such as non-ST elevated myocardial infarction (NSTEMI) and unstable angina (UA) are serious conditions that result from reduced blood perfusion to the cardiac tissue. Like STEMIs, these non-ST-segment elevated ACS (NSTE-ACS) require immediate attention and are exacerbated by excess sympathetic tone. In contrast to STEMIs, they may or may not be indicated for immediate percutaneous coronary intervention (PCI) due to the availability of less invasive therapies that may relieve the symptoms and begin to address the underlying perfusion deficit. These patients are classified into several risk categories that may then require invasive intervention at various timepoints: very high risk, high risk and intermediate risk categories with interventions at <2 hr, between 2 and 24 hr, and between 25 and 72 hr (<2 hr), respectively. Patients diagnosed as low risk may not receive invasive intervention and receive only medical and non-invasive strategies. Delivery of a therapy to help stabilize the cardiac system may prove beneficial to patients who are under observation for a NSTE-ACS such as NSTEMI or UA prior to referral to PIC.

In one configuration, the invention presents a method for improving the care of a patient under observation following diagnosis of NSTE-ACS prior to referral to PIC. The system and therapy can be delivered upon diagnosis in addition to other therapies, such as antithrombic therapies, to improve the care of the patient. Delivery of the therapy may further enhance the ability of the non-invasive therapies to treat the NSTE-ACS by allowing the non-invasive therapies more time to take effect prior to deterioration of the patient state such that they would require PCI. Such an effect may be the result of the reduced cardiac sympathetic tone, which can lead to improved perfusion of the cardiac tissue. Additionally, the therapy may prevent cardiac arrhythmia that may arise during the time prior to PCI, or upon incidence of STEMI.

This system may be comprised of a percutaneously deliverable lead that is then connected to an external pulse generator. The external pulse generator may be a small unit configured to be placed in contact with the patient, or a larger pulse generator that is placed at bedside. The pulse generator can then deliver therapy to the patient via the percutaneously placed lead while the patient is being monitored and risk level is established. In both configurations, the therapy can be ceased either upon referral to PCI or discharge from hospital, at which point the percutaneous lead can be removed from the patient. In another configuration, the system can be configured such that the patient continues receiving therapy after discharge, and the therapy ceased and leads removed at a later time.

Embodiments include systems and methods for preventing the formation of a chronic proarrhythmic cardiac and stellate substrate in patients hospitalized for acute myocardial infarction. In some embodiments, the devices, systems and methods described herein reduce the undesirable sympathetic tone in the extracardiac neural circuit to reduce, prevent, or reverse the undesired remodeling that occurs in the post-MI subacute and chronic settings.

A non-limiting example of the system includes an implantable neuromodulation system that delivers electrical currents to modulate the activity of a target nerve structure in the extracardiac autonomic system. The system is configured to deliver electrical current comprised of a waveform (including an ultra-low frequency (ULF) waveform or any of the waveforms described herein, other waveforms and non-electrical therapies), where the desired modulation of the target nerve activity is inhibition, and where the target nerve structure is the paravertebral sympathetic chain at thoracic levels T1-T2 (other nerves may be targeted in other examples). The system includes an electrical pulse generator and at least one electrical lead configured to safely deliver the waveform. The electrical leads are configured to be implanted percutaneously under image guidance, including ultrasound or fluoroscopic techniques, and configured to remain in electrical contact with the target nerve sufficient to modulate nerve activity. The electrical leads are configured to be removed without the need for surgical access at the conclusion of the therapy. The electrical pulse generator is configured to be placed externally and to connect with the electrical leads implanted percutaneously.

A non-limiting example of the system includes a modified paravertebral sympathetic chain at thoracic levels T1-T2, in which the sympathetic chain demonstrates reduced neural activity to reduce the cardiotoxic sympathetic tone. The modified paravertebral sympathetic chain also demonstrates reduced inflammation and glial activity.

A non-limiting example of the method to prevent formation of a chronic proarrhythmic cardiac and stellate substrate includes a surgical procedure to percutaneously place electrical leads in adequate proximity to the paravertebral sympathetic chain at thoracic levels T1-T2 to enable modulation of the neural activity. The electrical leads are then connected to an external electrical pulse generator. A stimulation signal is delivered via the leads and a response is observed to confirm that the lead has been positioned at the correct location. If not, the lead placement is adjusted, and another stimulation signal is delivered to confirm correct lead placement. Once correctly positioned, the electrical pulse generator is activated during the subacute post-MI timeframe to deliver a therapy to reduce the nerve activity in the target nerve structure, wherein the therapy is comprised of a waveform (e.g., ultra-low frequency waveform or other waveform). The reduced nerve activity reduces the cardiotoxic sympathetic tone for the duration of the subacute post-MI timeframe. The electrical pulse generator is deactivated at the completion of the therapy. The electrical leads are removed from the patient.

Study Showed Therapy Inhibits Sympathetic Tone

One preclinical study demonstrated the ability to use a ULF waveform to inhibit sympathetic tone. Application of sympathetic augmentation was blocked by application of ULF waveform in a reversable manner. Applying a ULF signal at the location described above caused a decrease in an adverse response to an adverse stimulant. During a first time period an adverse stimulant was applied. During a second time period both the adverse stimulant and an electrical nerve blocking signal (e.g., a ULF signal) were applied. The ULF signal was applied at the location described above.

Left ventricular pressure (LVP) did not change (% change=0) during about the first 50 seconds of observation. At about the 50 second mark, an adverse stimulant was applied, which immediately caused the LVP to increase, reaching an increase in pressure of about 20% within about 10 seconds. The ULF signal was then activated, which caused the LVP to decrease such that it exceeded the baseline value by only about 5%. When the ULF signal was removed (but the adverse stimulant remained), the LVP again began to increase.

However, once the adverse stimulant was removed, the LVP decreased back down to, the initial baseline value (of 0% change). Similar observations were made for the subject's heart rate and change in dP/dtₘₐₓ (change in maximum pressure over time).

Pain Management

Chronic pain is a significant burden on individuals and society as a whole. Nearly 50 million adults are estimated to have significant chronic or severe pain in the U.S. alone. Worldwide, chronic pain is estimated to affect more than 1.5 billion people. While surgical techniques are sometimes applied to remove a specific source of pain, typically due to impingement of a nerve, in many cases the precise cause of pain is not clear and cannot be reliably addressed via a surgical procedure. Pain management can alternatively be addressed by overwhelming the central nervous system with stimulating signals that prevent registration of pain inputs (gate control theory of pain). Typically, this stimulation in the case of spinal cord stimulation (SCS) is performed using metal electrodes and alternating current (AC) stimulation to produce these additional stimulating signals to prevent pain sensation. However, one major drawback is the presence of paresthesia, a sensation of tingling in the innervated region downstream from the stimulated nerve. Methods to eliminate paresthesia which patients can find discomforting have led to different means of stimulation from conventional tonic SCS (˜30-120 Hz) stimulation including high frequency stimulation (˜10 kHz) and burst stimulation (e.g., five pulses at 500 Hz delivered 40 times per second). (Tjepkema-Cloostermans et al, Effect of Burst Evaluated in Patients Familiar With Spinal Cord Stimulation, Neuromodulation, 2016 July 19(5):492-497).

An alternative means to manage pain signaling to the central nervous system is to prevent conduction of the pain signals from the peripheral signal source by directly blocking the pain signals as compared to masking the pain signals by generating alternative neural inputs to crowd out and inhibit pain signal transmission as in traditional SCS and gate theory. One means to do this is by applying a direct current (DC) to a nerve to prevent action potential (AP) generation and transmission. Because this does not stimulate the nerve as in traditional stimulation, paresthesia can be avoided. The mechanism leading to AP block has been attributed to a depolarization block that deactivates the sodium channels required for an action potential event under the electrode site. (See Bhadra and Kilgore, Direct Current Electrical Conduction Block of Peripheral Nerve, IEEE Transactions on Neural Systems and Rehabilitation Engineering, 2004 Sep. 12(3): 313-324).

Bhadra et al. showed that upon application of DC to nerve tissue, action potential conduction can be blocked (See Bhadra and Kilgore). The authors showed that removal of DC delivery from the same nerve tissue resulted in instantaneous restoration of nerve conduction. However, direct current has long been known to be dangerous to nerve tissue due to creation of toxic species at the electrode-nerve interface (Merrill, Electrical Stimulation of Excitable Tissue: Design of Efficacious and Safe Protocols, Journal of Neuroscience Methods, 2005, 141:171-198). Ackermann et al. and Fridman et al have developed systems and methods of safely delivering DC to nerve tissue by separating the toxic species created at the electrode interface from the nerve tissue (U.S. Pat. Nos. 9,008,800 and 9,498,621; Ackermann et al, Separated Interface Nerve Electrode Prevents Direct Current Induced Nerve Damage, J Neurosci Methods, 2011 September 201(1):173-176; Fridman and Santina, Safe Direct Current Stimulation to Expand Capabilities of Neural Prostheses, IEEE Transaction of Neural Systems and Rehabilitation Engineering, 2013 March 21(2):319-328; Fridman and Santina, Safe Direct Current Stimulator 2: Concept and Design, Conf Proc IEEE Eng Med Bio Soc, 2013: 3126-3129), each of the foregoing of which are incorporated by reference in their entireties. They also teach that rapid reversibility of nerve blockade is desirable and achievable through halting of DC delivery. Ackermann et al. teaches that an undesired, but reversible, suppression of nerve activity occurs with long term direct current delivery (where nerve tissue was shown to be non-conductive for a short period of time following cessation of DC delivery) (U.S. Pat. Nos. 9,008,800 and 9,498,621; Ackermann et al.), each of which are incorporated by reference in their entireties. Those authors specifically teach methods to reduce this suppression of nerve activity by limiting the duration of DC delivery to allow rapid nerve recovery upon cessation of DC delivery (e.g., within seconds) (U.S. Pat. Nos. 9,008,800 and 9,498,621; Ackermann et al.).

In some embodiments, the systems, devices, electrodes, leads, electrical waveforms and methods described herein are configured to achieve different effects, such as intentionally blocking nerve activity by using periodic DC pulses to intentionally place neural tissue in a state of hypersuppression without rapid reversibility upon cessation of DC delivery (reversibility that occurs in many minutes to hours, as opposed to seconds or less than a minute). Such systems and methods can inhibit neural activity through selective inhibition of antero-lateral column tissue in or near the spinal cord. Furthermore, such systems and methods are configured to inhibit nerve activity by inhibiting one or more peripheral nerves. With targeted nerve inhibition, signals from specific dermatomes and response in regional body sites can be managed.

Post-Myocardial Infarction Timeframe

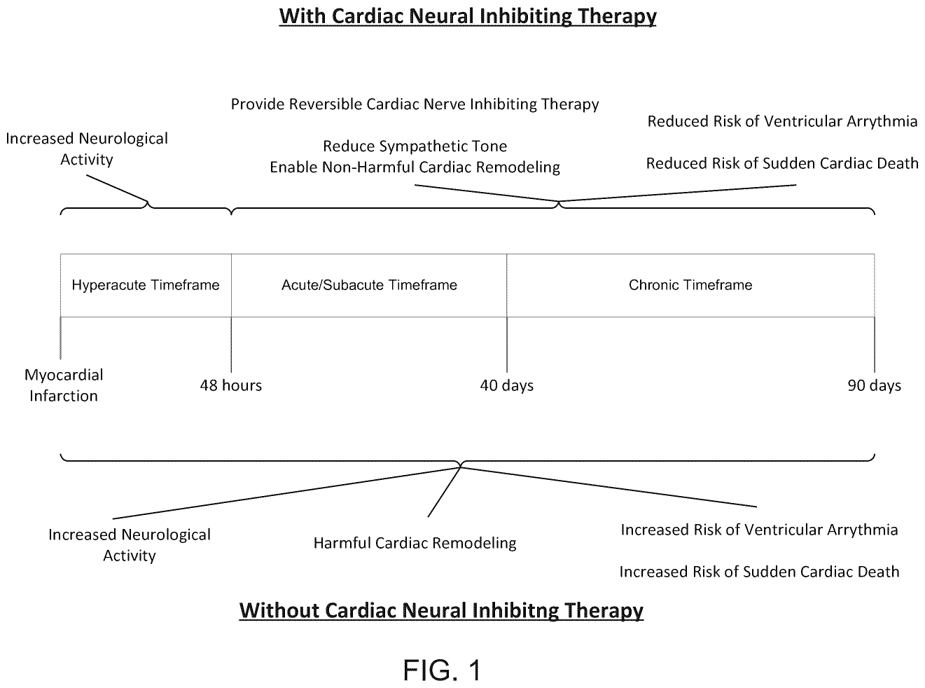

FIG. 1 is a timeline illustrating a post-myocardial infarction (MI) timeframe with and without cardiotoxic sympathetic inhibiting therapy and corresponding effects. After an event of cardiac distress (e.g., myocardial infarction), a patient's heart may undergo significant remodeling. Such remodeling may result in ventricular arrhythmias and sudden cardiac death (SCD). As shown in FIG. 1, the incidence of SCD as a result of ventricular arrhythmias can be broken down into three major timeframes: a hyperacute timeframe (typically lasting 48 hours from a myocardial infarction, or symptoms of an MI, although other periods less or more than 48 hours from MI can correspond to the hyperacute timeframe), a subacute/acute timeframe (typically 48 hours—40 days post MI or MI symptoms, although other periods can correspond to the subacute/acute timeframe), and a chronic timeframe (typically 40-90 days post MI or MI symptoms, although other periods can correspond to the chronic timeframe).

As disclosed herein, in a non-limiting example, reversible cardiac neural inhibiting therapy may be initiated for patients hospitalized for acute myocardial infarction within a critical period from MI or MI symptoms. The therapy inhibits neural activity thereby reducing sympathetic tone, and thereby prevents the formation of chronic proarrhythmic cardiac and stellate substrate in order to reduce infarct size and improve healing while reducing risk of subsequent adverse events.

As related to cardiac nerve blocking therapy, the depth of blockade or inhibition may refer to the extent to which neural activity is reduced. For example, cardiac nerve blocking therapy (sometimes referred to as cardiac neural or nerve inhibiting therapy) may be delivered such that the blocking depth is capable of one or more of the following: a complete block of neural activity in the target structure, a partial block of neural activity in the target structure, a combination of complete and partial block of neural activity in which a complete block is achieved for a certain period of time or in response to an event or measurement and a partial block is achieved for a certain period of time or in response to an event or measurement, a complete block for which the therapy duty cycle is 100%, a complete block for which the therapy duty cycle is <100%, such that the therapy is active for a predefined or determine in-vivo time period to provide adequate therapy, a partial block for which the therapy duty cycle is 100%, a partial block for which the therapy duty cycle is <100%, such that the therapy is active for a predefined or determine in-vivo time period to provide adequate therapy.

In some embodiments, as shown in FIG. 1, delivering cardiac neural inhibiting therapy occurs within the subacute/acute timeframe. In the illustrated embodiment, therapy is initiated 48 hours post-MI. However, in other embodiments, therapy may be initiated as soon as the patient is available to receive therapy, such as during the hyperacute timeframe. In some embodiments, delivery of the cardiac neural inhibiting therapy may be initiated within 14 days of the MI event, within 2 days of the MI event, before 14 days of the MI event, or during the MI event.

In some embodiments, before delivery of cardiac neural inhibiting therapy, a therapy device is deployed. For example, in some embodiments, the therapy device is deployed upon EMS (emergency medical service, ambulance, clinician) arrival to the scene of suspected MI, during EMS transport of the patient to the care facility, upon admission of the patient to the care facility prior to diagnosis of MI, after diagnosis of STEMI but prior to reperfusion (OR or Rx) (e.g., if catheter lab not available, prior to onset of thrombolysis), within the setting for reperfusion (e.g., before reperfusion has taken place, while reperfusion is underway, or after reperfusion has taken place), after patient returns for post-operative monitoring, prior to patient discharge after reperfusion, after patient has been transferred to a central telemetry unit, after patient has been transferred to an in-patient hospital setting, after patient has been discharged and returns for follow-up, after diagnosis of NSTEMI but prior to discharge or reperfusion, while patient is being monitored following diagnosis of NSTEMI, or after patient has been referred for reperfusion following NSTEMI diagnosis but before reperfusion has occurred.

Onset of cardiac neural inhibiting therapy may occur at various times within a given timeframe. For example, in some embodiments, delivery of the cardiac neural inhibiting therapy may occur immediately following placement to assess proper placement, shortly after placement in the hyperacute setting (e.g., within 48 hours), after placement in the acute setting (e.g., between 48 hours and 7 days), after placement in the subacute setting (e.g., between 7 days and 40 days), after placement in the chronic setting (e.g., after 40 days), on-demand following patient daily life activities, following a pre-defined schedule set by the physician, patient, or other party (e.g., activated in the morning when circulating catechoalmines rise rapidly), or in response to detection of a biomarker or sensed parameter.

As illustrated in FIG. 1, resulting effects on the heart where cardiac neural inhibiting therapy is provided are shown above the post-myocardial infarction timeline. In contrast, resulting effects on the heart where cardiac neural inhibiting therapy is not provided are shown below the post-myocardial infarction timeline. In some embodiments, timing and delivery of the cardiac nerve blocking therapy is connected to the post-myocardial infarction timeline.

In one embodiment, the hyperacute timeframe occurs within 48 hours post-MI symptoms. During this timeframe, hyperacute arrhythmias may be a direct result of ischemic tissue.

In one embodiment, the subacute/acute timeframe is between 48 hours and 40 days post MI symptoms. During this timeframe, a patient's nervous system may be hyperactivated. This elevated sympathetic activity may create new reflex pathways—causing immediate burden on the heart and asynchronous pathways of electrical conduction (increasing risk of arrythmia).

As illustrated in FIG. 1, without cardiac neural inhibiting therapy, a patient's heart is subject to increased sympathetic tone. In addition, a patient's heart may be subject to harmful remodeling during such increased sympathetic tone. During this timeframe, cardiac tissue is actively being remodeled to recover from the MI event.

Without cardiac neural inhibiting therapy, increased asynchronous pathways may lead to cardiac tissue that is stiffer and less electrically conductive. However, with cardiac neural inhibiting therapy, this detrimental effect may be avoided. For example, cardiotoxic sympathetic inhibiting therapy delivered during the subacute/acute timeframe can prevent the formation of proarrhythmic cardiac substrate and paravertebral chain.

In one embodiment, the chronic timeframe occurs between 40 and 90 days post MI symptoms. During this timeframe, there is a significant increase in ventricular arrhythmias as compared to the subacute/acute phase. Reduction in infarct size and non-harmful remodeling resulting from cardiac neural inhibiting therapy during the subacute/acute timeframe may reduce arrhythmia incidence during the chronic timeframe. As such, there is a decreased risk of ventricular arrythmia and SCD during the chronic timeframe with the cardiac neural inhibiting therapy.

In some embodiments, therapy cessation may occur within the acute setting (e.g., between 48 hours and 7 days), within the subacute setting (e.g., between 7 days and 40 days), within the chronic setting (e.g., after 40 days). In some embodiments, therapy cessation may occur after physiological assessment indicating completion of therapy, such as in response to one or more of: infarct assessment, cardiac output assessment, left ventricle function (e.g., LVEF), arrhythmia incidence, electrophysiology assessment (e.g., heart rate variability, electrophysiologic heterogeneity, monomorphic VT inducibility), assessment of biomarkers (e.g., catecholamines, troponin, galanin, nerve growth factor), assessment of nerve density, assessment of paravertebral sympathetic chain neural activity, assessment of paravertebral sympathetic chain inflammation, assessment of paravertebral sympathetic chain glial activity, assessment of electrophysiology to sympathetic stimulation (e.g., repolarization), upon direction of the overseeing physician, due to onset of undesirable complications, on-demand following patient daily life activities, following a pre-defined schedule set by the physician, patient, or other party, or in response to detection of a biomarker or sensed parameter.

In some embodiments, therapy device removal may occur at various timeframes and for various reasons. For example, in some embodiments, therapy device removal may occur within the acute setting (e.g., between 48 hours and 7 days), within the subacute setting (e.g., between 7 days and 40 days), or within the chronic setting (e.g., after 40 days). In some embodiments, therapy device removal may occur upon direction of the overseeing physician, due to onset of undesirable complications, due to the need for another implantable device (e.g., ICD), or even after physiological assessment indicating completion of therapy.

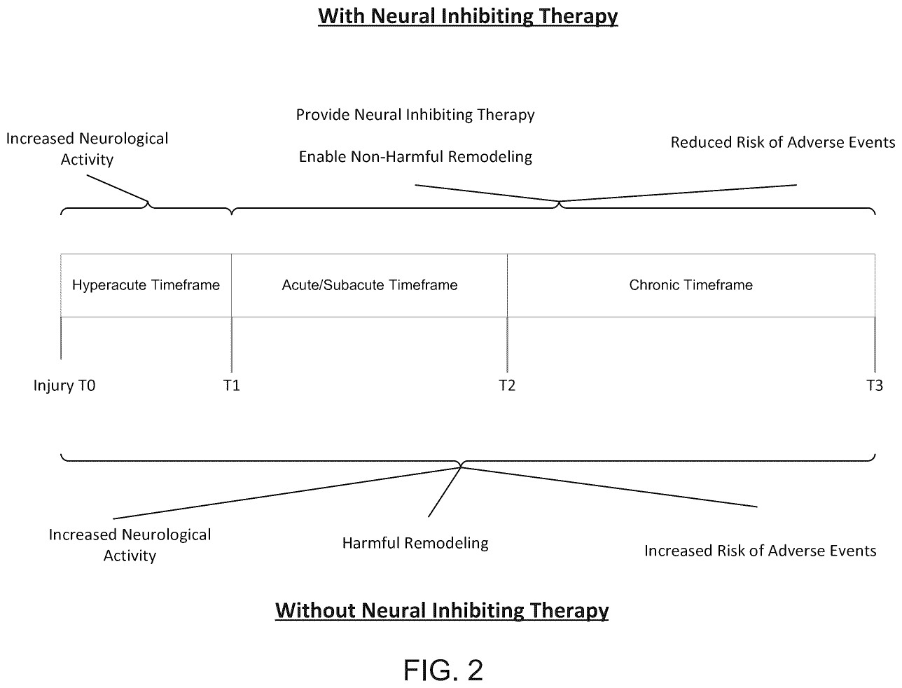

FIG. 2 illustrates an embodiment of a timeline for reducing risk of an adverse event following injury to a patient, where neural inhibiting therapy has been provided. The timeline also indicates (below the timeline) the harmful effects and increased risks that would otherwise occur if neural inhibiting therapy is not provided. In the illustrated timeline, an injury occurs at an initial time T0. Increased neurological activity may occur in response to the injury. For example, when the injury is a cardiac injury, the resulting increased neurological activity may include increased sympathetic and/or parasympathetic tone. Other injuries and other neurological responses may occur.

During a critical time-period post injury, a neural inhibiting therapy is applied. The neural inhibiting therapy may include any of the electrical stimulation therapies or non-electrical therapies described herein. The neural inhibiting therapy is provided to target a nerve or nerves associated with the increased neurological activity resulting from the injury. In the illustrated embodiment, the neural inhibiting therapy is initiated during a subacute/acute timeframe that begins a time T1 after the injury. In other embodiments, the neural inhibiting therapy may be initiated prior to T1 or after T1.

The neural inhibiting therapy continues for a predetermined time period, until a monitored physiological parameter indicates that the injury has been healed, or until any of the therapy-ending events described herein occurs. In the illustrated embodiment, the neural inhibiting therapy continues until the end of a chronic timeframe T3. The chronic timeframe begins at a time T2 when the subacute/acute timeframe ends.

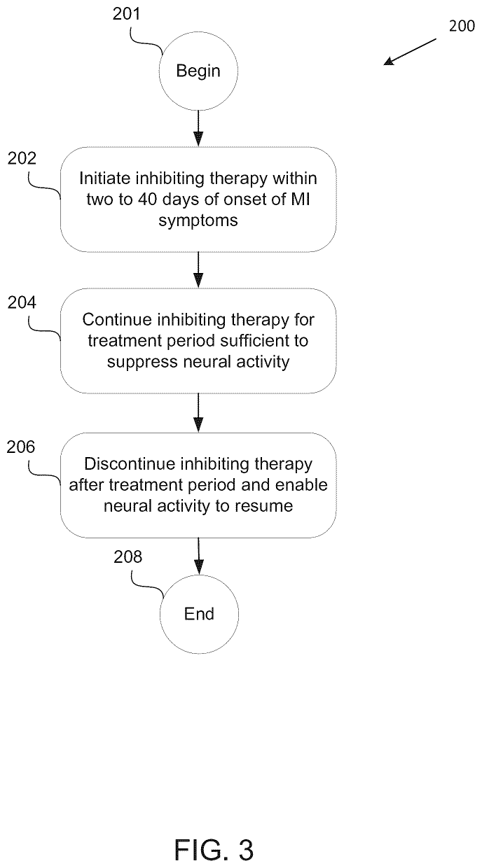

FIG. 3 is a flow diagram illustrating an example method 200 for providing cardiac neural inhibiting therapy to inhibit neural activity to prevent the formation of unfavorable sequelae. For example, such neural inhibiting therapy may be provided to prevent formation of proarrhythmic cardiac and stellate substrates. Such method may also be provided to reduce sympathetic tone. In some embodiments, the method 200 may be implemented by any system configured to provide cardiac nerve blocking therapy described herein.

The method 200 begins at block 201. At block 202, the therapy system initiates delivery of inhibiting therapy after symptoms of acute coronary syndrome occur. For example, therapy may be inhibited after the onset of MI symptoms, or any other symptom of acute coronary syndrome. In the illustrated embodiment, therapy is initiated within 40 days of onset of acute coronary syndrome symptoms. However, therapy may be initiate in other time windows, such as two to 40 days within onset of acute coronary syndrome symptoms, or 40 days or more following the onset of acute coronary syndrome symptoms.

In some embodiments, the inhibiting therapy includes delivering an electrical therapeutic signal to a nerve sufficient to substantially prevent, block, inhibit, or reduce neural activity of the nerve. In some embodiments, delivering therapy occurs within 14 days of the symptoms, within 2 days of the symptoms, before 14 days of the symptoms, or during the symptoms.

In some embodiments, delivering therapy includes delivering therapy to a nerve associated with the extracardiac sympathetic nervous system of the patient. For example, an electrical inhibiting current to a paravertebral chain at thoracic levels T1-T2, stellate ganglia, spinal cord, ansa subclavia or dorsal root ganglia.

In some embodiments, delivering therapy comprises one or more of delivering a blocking electrical waveform, delivering an ultra-low frequency waveform, providing a non-electrical therapy, providing thermal energy, providing magnetic energy, or providing ultrasonic energy, or a providing a combination of such therapies.

In some embodiments, the method 200 also includes implanting an electrical contact close to the nerve associated with the extracardiac sympathetic (and/or parasympathetic) nervous system of the patient. In some embodiments, implanting includes implanting the electrical contact under ultrasound or fluoroscopy or other image guidance method.

At block 204, the therapy system continues delivery of inhibiting therapy to nerve for a treatment period sufficient to inhibit neural activity.

In some embodiments, continuing inhibiting includes inhibiting neural activity for at least 14 days, up to 40 days, until a sympathetic tone is decreased by a desired amount in the absence of the neural inhibiting, until a biomarker level of a biomarker relating to myocardial death drops by a desired amount. For example, in some embodiments, the biomarker is troponin or any other biomarker described herein.

At block 206, therapy system discontinues delivery of neural inhibiting therapy after the treatment period to enable neural activity resume. In some embodiments, deactivating blocking comprises deactivating a waveform generator.

In some embodiments, method 200 further includes removing a treatment device from the patient, wherein the treatment device includes an external pulse generator configured to generate a nerve inhibiting waveform and a percutaneous lead having one or more electrodes configured to deliver the nerve inhibiting waveform to a target nerve or nerves. For example, in some embodiments, discontinuing inhibiting therapy includes removing an implantable lead or removing a patch or other device from a skin surface of a patient.

In some embodiments, the method 200 may be performed by any cardiac nerve inhibiting therapy device. The method 200 ends at block 208.

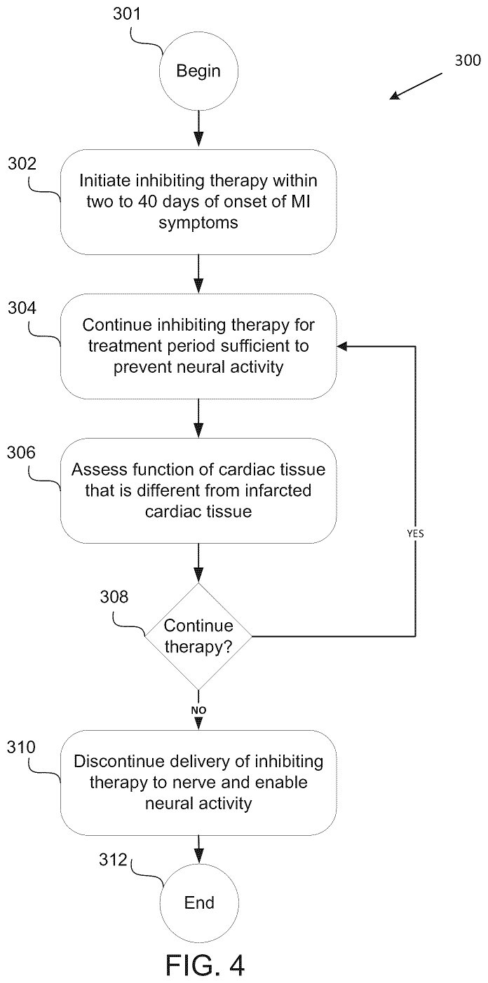

FIG. 4 is a flow diagram illustrating an example method 300 for preventing the formation of unfavorable sequelae. For example, such a method may be provided to prevent formation of proarrhythmic cardiac and stellate substrates. Such method may also be provided to reduce a sympathetic tone in an extracardiac neural circuit. In some embodiments, the method 300 may be implemented by any of the neural inhibiting therapy systems described herein.

The method 300 begins at block 301. At block 302, the therapy system initiates delivery of neural inhibiting therapy to a nerve within 40 days of onset of acute coronary syndrome symptoms, such as, but not limited to MI symptoms. Other time windows for initiating therapy may be utilized in other embodiments, as described in greater detail herein. For example, therapy may be initiated within two to 40 days of onset of symptoms or 40 days or more after onset of symptoms. In some embodiments, the neural inhibiting therapy includes delivering an electrical therapeutic signal to a nerve sufficient to substantially inhibit, block, reduce, or prevent neural activity of the nerve.

In some embodiments, delivering therapy occurs within 14 days of symptoms, within 2 days of symptoms, before 14 days of symptoms, or during the symptoms.

In some embodiments, delivering therapy to the nerve comprises delivering therapy to a paravertebral chain at thoracic levels T1-T2, stellate ganglia, spinal cord, ansa subclavia or dorsal root ganglia.

In some embodiments, delivering therapy comprises one or more of delivering an inhibiting electrical waveform, delivering an ultra-low frequency waveform, providing a non-electrical therapy, providing thermal energy, providing magnetic energy, providing ultrasonic energy, or providing a combination of such therapies.

In some embodiments, the method 300 further includes implanting (e.g., percutaneously delivering) an electrical contact close to one or more nerves associated with the extracardiac sympathetic and/or parasympathetic nervous system of the patient. In some embodiments, implanting comprises implanting the electrical contact under ultrasound or fluoroscopy or other image guidance method.

At block 304, the therapy system continues delivery of neural inhibiting therapy to a nerve to inhibit neural activity.

In some embodiments, continuing inhibiting therapy comprising inhibiting neural activity for at least 14 days, up to 40 days, until a sympathetic tone is decreased by a desired amount in the absence of the neural inhibiting, until a biomarker level of a biomarker relating to myocardial death drops by a desired amount. For example, in some embodiments, the biomarker is troponin or any other biomarker described herein.

At block 306, a function of cardiac tissue is assessed. If, at block 308, the assessment indicates that neural inhibiting therapy should be continued, the method returns to block 304. If the assessment indicates that therapy should be discontinued, the method continues to block 310.

At block 310, the therapy system discontinues delivery of neural inhibiting therapy to and enables neural activity to resume. In some embodiments, deactivating blocking comprises deactivating an electrical waveform generator. The method 300 ends at block 312.

In some embodiments, the method 300 further includes removing a treatment device from the patient. For example, in some embodiments, discontinuing comprises removing an implanted, removing a patch, and/or removing another device from a skin surface of a patient.

In some embodiments, the systems and methods described may be implemented by the therapy system. As noted above, the therapy system may be any system configured to provide cardiac nerve inhibiting therapy. For example, in some embodiments, any neuromodulation therapy that results in a decreased neural output may be incorporated into the systems and methods described herein.

In some embodiments, the therapy system may include any electrical, ultrasound, thermal, magnetic, or drug system configured to provide nerve inhibiting therapy. For example, in some embodiments, an ultrasound therapy system may provide nerve inhibiting therapy through heating and/or cooling (e.g., sequentially) of the neural target. In some embodiments, a magnetic therapy system may provide nerve inhibiting therapy through alternating and direct magnetic fields. In some embodiments, a drug-based therapy system may provide nerve inhibiting therapy through continuous infusion of neural inhibitory compound, single injection of neural inhibitory compound, or a drug-eluting device/compound.

In some embodiments, surgical approaches may be used to deliver a system capable of modulating neural activity in a target nerve. For example, alternative surgical approaches may include: targeting the paravertebral sympathetic chain, such as via endovascular lead placement, surgical lead placement (e.g., thoracic surgery, VATS, thorascopic surgery, sternotomy, thoracotomy, spinal surgery), transcutaneous placement, percutaneous placement, targeting the spinal cord/dorsal roots, epidural placement, transcutaneous placement. In some embodiments, in any of the preceding surgical approaches, the therapy may be delivered unilaterally, on the left/right side. In some embodiments, in any of the preceding surgical approaches, the therapy may be delivered bilaterally.

In some embodiments, electrical therapy systems may be used to provide cardiac nerve blocking therapy as described by the system and methods above. In one embodiment, an electrical therapy system may include a pulse generator and an electrical lead that includes one or more electrodes.

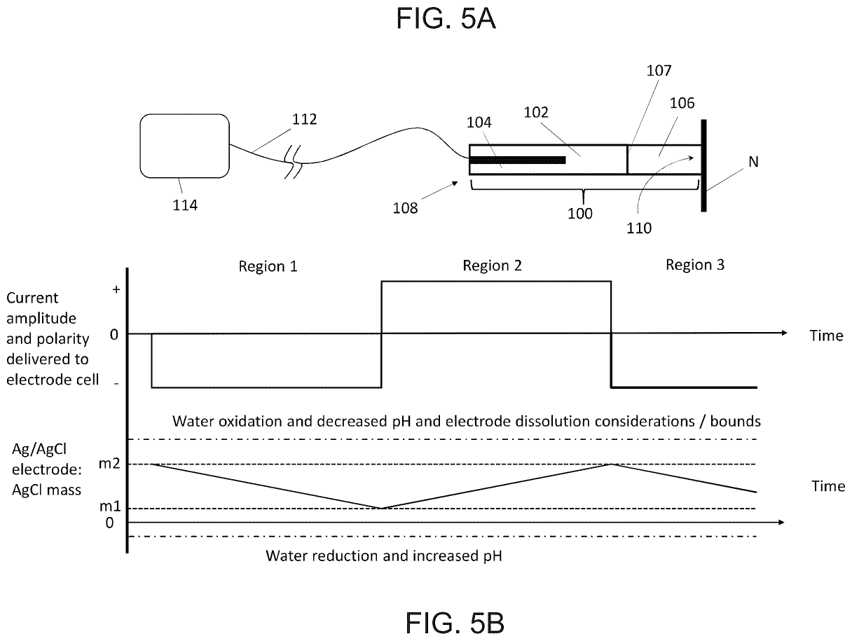

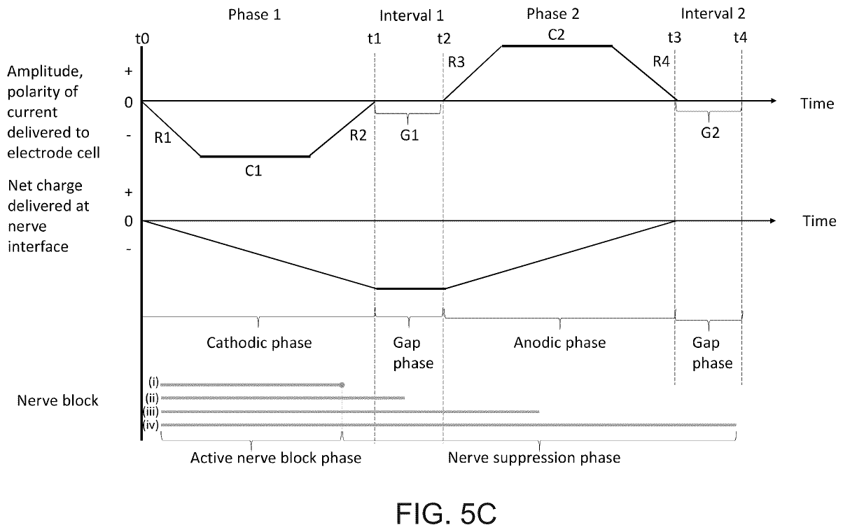

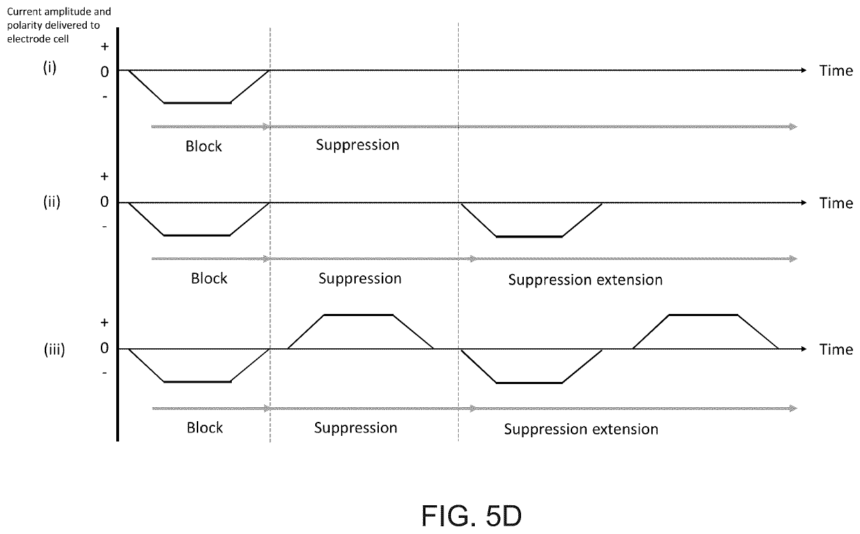

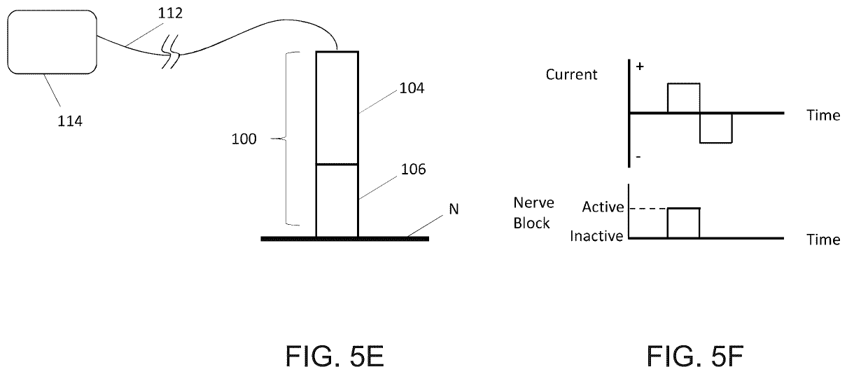

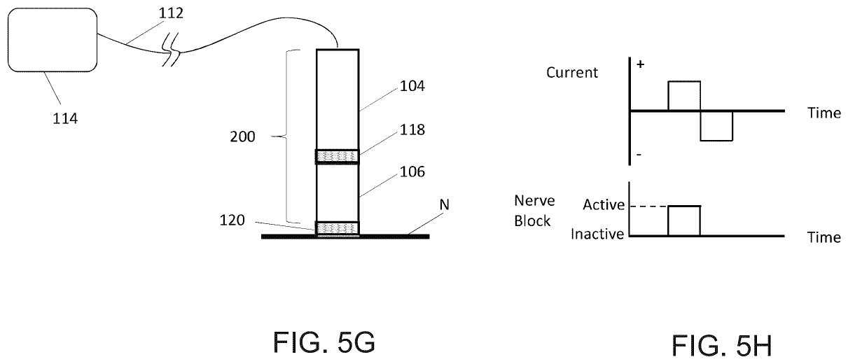

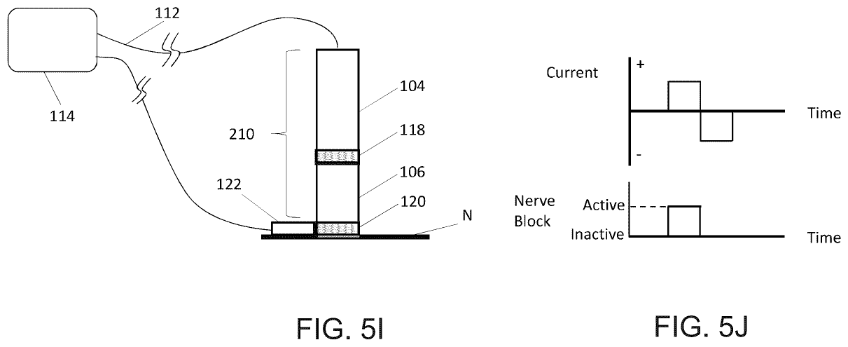

One embodiment of a therapeutic system including a ion generating electrode is illustrated in FIG. 5A. Electrodes where current in the form of ions is generated proximal to the at least one target nerve may include an ionically conductive material such as a liquid (e.g., saline or other electrolyte solution), gel, hydrogel, hydrocolloid, polymer, or film. In an alternative embodiment, the ionically conductive materials may be separated by a screen or other filter or membrane material from the nerve tissue. This separating interface may be configured to selectively allow ions through to the nerve to reduce nerve damage such as microporous screens, non-woven screens, ion-exchange membranes (IEM), supported liquid membranes or ionogels, polymer electrolytes such as polyethylene oxide (PEO), polypropylene oxide (PPO), polyvinylidene fluoride-co-hexafluoropropylene copolymer (PVDF-HFP), solid ion conductors, and ion-selective films including cation exchange membranes and anion exchange membranes.

The nerve-interfacing element of the electrode may be further configured to be exposed selectively along the electrode and may be otherwise insulated from the nerve by an ionically impermeable layer. The impermeable layer may also be configured to be electrically insulating to current.

The ionically conducting material may also be separated into multiple regions which may contain different types of ionically conducting material. The interfaces between the different regions may be delineated by semi-permeable membranes or screens that allow for selective or general ionic flow but limit the passage of damaging by products from the conversion of electron current to ionic current. This separating element may be configured to selectively allow ions through to the nerve to reduce nerve damage such as microporous screens, non-woven screens, ion-exchange membranes (IEM), supported liquid membranes or ionogels, polymer electrolytes such as polyethylene oxide (PEO), polypropylene oxide (PPO), polyvinylidene fluoride-co-hexafluoropropylene copolymer (PVDF-HFP), solid ion conductors, and ion-selective films including cation exchange membranes and anion exchange membranes. The different ionically conducting materials may also take different forms. As an example, the nerve may be in contact with a hydrogel which is in contact with a liquid such as an electrolyte solution which then is in contact with a traditional electrical current electrode material.

In some embodiments the traditional electrode may be made from a material such as platinum, platinum-iridium, carbon, titanium nitride, copper, tantalum, silver, silver-chloride or other metals and materials or combinations thereof. In some embodiments, the traditional electrode may be made from carbon, graphite, glassy carbon, dendritic carbon, or other conductive materials. By using high-charge chemistry amplitude and duration of direct current (DC) block can be increased. Candidate chemistries include using a combination of Ag/AgCl electrode in an electrolyte bath (or other suitable ionically conductive material) such as saline that is in ionic contact with neural tissue of interest. In some embodiments the electrode is reversible and can be restored to its initial state. In some embodiments the electrode is sacrificial and the electrochemical reaction that occurs at the electrode cannot be reversed to restore the electrode to its initial state.

The combination of traditional electron-carrying electrode material and ionic conducting material and the conversion mechanism can be collectively characterized as an electron-ion current conversion cell (EICCC). One such example might be a silver/silver chloride (Ag/AgCl) electrode immersed in a saline, e.g., isotonic 0.9% NaCl saline solution fluidly in contact with a saline-containing hydrogel. Upon driving of an electric current through the conventional electrode, reduction of the solid AgCl will drive conversion to solid Ag and Cl− ion formation generating a flow of ions or an ion current. This ionic current flow can be used to modulate the nerve membrane potential and, for example, inhibit nerve conduction.

The membrane potential may be modulated in such a manner that the potential is ramped up slowly enough to avoid action potential generation as the nerve tissue is depolarized. Upon reversal of the electric current, oxidation of the previously formed Ag or other Ag in the Ag/AgCl electrode will be oxidized to generate AgCl deposits on the electrode, driving the ion current in the opposite direction. Due to the extremely low solubility of Ag and AgCl in saline, the electrode remains mechanically intact during forward and reverse current delivery. In combination, the reduction-oxidation reactions create a fully reversible EICCC.

To maintain the preferred reduction-oxidation reaction between Ag and AgCl (or other electrode materials), the amount, e.g., mass, volume, density, or another parameter of the AgCl on the electrode may be maintained within 5%-95%, 10%-90%, 20%-80%, 25%-75%, 30%-70% of its original starting mass on the electrode to ensure that the AgCl is never depleted or saturated, enabling other deleterious reactions from happening at the electrode. In some embodiments, the amount of the electrode can be maintained at least about, or no more than about 5%, 10%, 15%, 20%, 25%, 30%, 35%, 40%, 45%, 50%, 55%, 60%, 65%, 70%, 75%, 80%, 85%, 90%, 95%, 100%, or ranges of between about any two of the foregoing values. In other words, the electrode is reversible and can be restored to its original or substantially to its near-original state.

To generate more surface area for the electrochemical reactions to occur, the traditional electrodes may be made from high surface area to volume structures such as roughened surfaces, woven surfaces, patterned surfaces, reticulated foam structures, porous sintered bead structures, nano- or micro-patterned structures to expose additional material surface area. High-charge chemistry electrodes can be biocompatible, or suitably sequestered from body if not. A high surface area electrode material (e.g., Ag/AgCl) in the EICCC may be utilized specifically to decrease the electrode potential drop or to reduce the increase in electrode potential drop which may occur with prolonged current delivery. In some embodiments the EICCC driving current may be between about 0 mA and about 1 mA, between about 1 mA and about 2 mA, between about 2 mA and about 4 mA, between about 4 mA and about 8 mA, higher than about 8 mA, about 0.5 mA, 1 mA, 2 mA, 3 mA, 4 mA, 5 mA, 6 mA, 7 mA, 8 mA, 9 mA, 10 mA, or ranges incorporating any two of the foregoing values. In some embodiments this driving current is then used to generate a corresponding ionic current of similar magnitude, depending on the specific electrochemical reactions.

Another embodiment of the EICCC may comprise a material such as tantalum or titanium nitride to generate a capacitive traditional electrode interface instead of an interface at which an electrochemical reaction occurs. Transparent conducting oxides (TCOs) such as fluorine-doped tin oxide (FTO), nickel titanium dioxide (Ni/TiO2), and other titanium dioxide (TiO2) constructs are also candidate materials that have high charge carrying capacities. In this configuration, charge generation at the traditional electrode surface would attract ionic species from the ionically conductive material until the charge at the traditional electrode interface is passivated.

Charging of the capacitive material with an electric current of one polarity can generate current flow in the form of ions. Reversing the polarity of the current flow to the capacitive material can effectively reset the system for a subsequent charging to generate further ionic current flow. To generate more surface area for increased ion current flow capacity to occur, the traditional electrodes may be made from high surface area to volume structures such as roughened surfaces, reticulated foam structures, porous sintered bead structures, nano- or micro-patterned structures to expose additional material surface area. In one embodiment, this capacitive structure is in fluid contact with an electrolyte solution that contacts an electrolyte-saturated hydrogel in contact with target nerve tissue to enable ion current flow to the tissue.

In a further embodiment of the EICCC, a combination of both electrochemical and capacitive mechanisms may be used to convert current in the form of electrons to current in the form of ions.

To deliver ionic current to the nerve to inhibit nerve function, the traditional electrode may be connected via a conductive lead to one or more current sources. A single nerve-electrode interface can inhibit nerve activity when current is applied in one polarity to the EICCC (inhibiting phase). When the current polarity is reversed to return the electrode to its original state (which may be a non-inhibiting phase or also an inhibiting phase), the nerve may or may not continue to inhibit stimuli from passing along the nerve. If the nerve has been placed into a state of hypersuppression, the nerve will continue to prevent AP propagation and inhibit signal propagation regardless of the phase state of the electrode. Fridman and Santina have described a means to enable continuous block when current polarity is reversed as driven by an alternating current (AC) using a series of valves to direct current flow direction (Fridman and Santina, Safe Direct Current Stimulation to Expand Capabilities of Neural Prostheses, IEEE Transaction of Neural Systems and Rehabilitation Engineering, 2013 March 21(2):319-328; Fridman and Santina, Safe Direct Current Stimulator 2: Concept and Design, Conf Proc IEEE Eng Med Bio Soc, 2013: 3126-3129). However, in some cases it is desirable to have a simpler system which does not require the use of valves, which present additional failure points and add bulk to an implantable system. A simpler, more robust system may be configured without valves and such moving parts by using multiple EICCCs to provide constant stimulation of the nerve tissue itself. In one embodiment to provide continuous inhibition, two nerve-electrode interfaces are present and connected to one or more current sources.

The first nerve-electrode interface EICCC is run with the current in one polarity to inhibit nerve activity while the second nerve-electrode interface EICCC is run with the opposite polarity. After a period of time, the current polarities of the first and second EICCCs are reversed and the second nerve-electrode interface inhibits nerve activity while the first nerve-electrode interface EICCC state is reversed to its prior state. By cycling the dual-EICCC electrode currents, continuous nerve inhibition can be maintained at the target nerve. As can be appreciated, more than two, such as 3, 4, 5, 6, 7, 8, 9, 10, or more EICCCs may also be used to facilitate the same continuous nerve inhibition. Electrodes may also be run in either monopolar or bipolar configurations. In some embodiments the EICCC system is configured to not have any mechanically moving parts such as valves or hinges.

Alternatively, nerve activity may be suppressed which means that nerve activity remains inhibited even after removal or discontinuation of the inhibiting current. The nerve may be further put into a state of hypersuppression in which the nerve remains inhibited without rapid reversibility after cessation of DC delivery. Modulation of the initial current delivered to the nerve tissue including ramp rate, current amplitude, total charge delivery, and waveform shape can be used to place the nerve in a state of suppression. During the state of suppression, the EICCC may be returned to its initial state by reversing the current polarity used to generate the initial inhibition and suppression state. During the period of reverse current flow, the nerve may remain in a state of hypersuppression. In another configuration the EICCC may deliver subsequent inhibiting current inputs that extend the suppression duration, with periods of no current delivery, or of reversal current in between inhibition current doses. The nerve tissue may remain in a state of hypersuppression during the periods of non-inhibition current input. In another configuration, the EICCC may be configured to deliver subsequent current inputs on a schedule.

In some embodiments, the DC inhibiting waveform may have an amplitude of between 0-250 microamps, 250-500 microamps, 500-1000 microamps, 1000-1500 microamps, or 2000 microamps, or higher, or about, at least about, or no more than about 50, 100, 150, 200, 300, 400, 500, 600, 700, 800, 900, 1,000, 1,100, 1,200, 1,300, 1,400, 1,500, 1,600, 1,700, 1800, 1,900, 2000 microamps or more, or other ranges incorporating any two of the aforementioned values. Placing a nerve into a state of hypersuppression may be facilitated in some embodiments by delivering a charge of 10-50 millicoulombs, 50-100 millicoulombs, 100-500 millicoulombs, 500-1000 millicoulombs, or 1000 millicoulombs or greater, or about, at least about, or no more than about 10, 25, 50, 100, 200, 300, 400, 500, 600, 700, 800, 900, 1,000, or more millicoulombs, or other ranges incorporating any two of the aforementioned values, and depending on nerve size and desired hypersuppression duration. DC block amplitude and current duration may be tuned to enable hypersuppression in the range of, for example, 0-0.5 times the duration of initial DC inhibition, 0.5-1 times the duration of initial DC inhibition, 1-1.5 times the duration of initial DC inhibition, 1.5-2 times the duration of initial DC inhibition, and greater than 2 times the duration of initial DC inhibition, or about, at least about, or no more than about 0.1×, 0.2×, 0.3×, 0.4×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.5×, 3×, 4×, 5×, or more relative to the duration of initial DC inhibition, or ranges including any two of the aforementioned values.

Sensing the local state of and proximal to the nerve tissue can also provide a useful measure for determining when to provide current inputs to extend nerve suppression as well as to provide a feedback loop for initial current delivery to generate the initial nerve inhibition by modulating the nerve potential such that it cannot transmit action potentials. In one embodiment the nerve's ability to conduct action potentials is monitored such that as direct current is delivered to the nerve tissue, the direct current delivery can be maintained to ensure that the nerve inhibition is maintained, for example. Nerve conduction ability may be monitored by any suitable measure such as delivering a stimulus pulse and measuring compound action potential signal.

In some embodiments sensing is in the form of a reference electrode to measure potential differences relative to the two electrodes which are passing the active current. In some embodiments the active current is modulated in response to one or more measured electrode potentials relative to the reference electrode. In some embodiments the active current is modulated when measured electrode potential indicates that undesired electrochemical reactions may occur at one or more active electrodes. For example, active current may be reduced or ceased upon measurement of an active electrode potential that indicates water electrolysis is occurring or possible. The EICCC may be operated with a direct current input or by applying a potential difference between the working electrode and an auxiliary or counter electrode. In some embodiments, a reference electrode may be located within the EICCC or at the distal end of the EICCC proximal to the nerve tissue.

FIG. 5A shows an embodiment of an EICCC electrode 100 in which an electrode 104 is immersed in an electrolyte solution 102 which in is contact with an ion-conductive material-electrolyte solution interface 107 with an ion-conductive material 106 such as a fluid, hydrogel, gel or other polymer that electrically contacts the nerve tissue N or area proximal to the nerve tissue N. The EICCC electrode 100 also comprises an electrically insulated, biocompatible enclosure 108 housing the traditional electrode 104, electrolyte 102, and biocompatible ion conducting material 106 with an aperture (near 110) to enable electrical contact with the nerve N or area proximal to the nerve tissue N. The system further comprises a current delivery lead 112 between the current source 114 and the electrode 104. The current source 114 may be located external or internal to the body depending on the application need. An exemplary non-limiting embodiment of the EICCC 100 comprises a silver, silver-chloride (Ag/AgCl) electrode in a 0.9% saline solution in fluid contact with an electrolyte saturated hydrogel (agar preparation with 0.9% saline).

With an Ag/AgCl electrode used to generate current via reduction of the AgCl on the electrode in a saline solution (NaCl), a sustainable and reversible electrochemical reaction can be achieved to convert current in the form of electrons into current in the form of ions. As seen in Region 1 of FIG. 5B, by pushing a constant current from the current source, the mass of the AgCl electrode will decrease from mass m2 to mass m1 during a cathodic current (reduction reaction) and then increase as seen in Region 2 from mass m1 back to mass m2 with an anodic current during an oxidative reaction. Furthermore, it can be appreciated that by limiting the maximum mass of the AgCl to m2 such that the mass of unreacted Ag is greater than zero helps prevent the Ag/AgCl reaction from depleting available Ag for the electrochemical reaction and provides a reserve safety factor in the event of excess current delivery similar to maintaining m1 above zero. In Region 3, the current polarity is again reversed to match that of Region 1. By not depleting the AgCl mass to zero, the preferred reaction between conversion of solid AgCl to solid Ag with generation of chlorine ions and vice-versa:

AgCl(s)+e⁻⇔Ag(s)+Cl⁻

Because water electrolysis or hydrolysis happens at higher reduction potential than AgCl, AgCl dissolution will be preferred preventing undesired reactions and generation of OH−, H+ or oxygen free radicals in the EICCC. Further notable is that the absolute value of the area between the current amplitude and the x-axis (time) can be used to define the total charge delivered (or removed) from the electrode to allow for determination or prediction and/or control of the electrode AgCl mass. It will be appreciated that the current waveform shapes in the different regions need not be perfect square waves but may include finite slopes that ramp from zero amplitude to their final maximum amplitude as well as from their maximum waveform amplitude back to zero current. Waveforms may also be non-linear in pattern and may vary between regions. In one embodiment, the total charge delivered in Region 1 is equivalent to the total charge removed in Region 2. In other words, the magnitude of the area below the current waveform in Region 1 is the same as that of Region 2. Different regions may also be spaced apart in time by a period of zero current (not shown) in which the AgCl electrode mass is conserved while no current is being delivered.

The silver-silver chloride system offers several potential benefits over other electrochemical reactions. For example, the standard potential of the silver-silver chloride reaction is about 0.22 V, which is advantageously well below the voltage at which electrolysis occurs. Electrolysis can occur when the magnitude of potential or voltage used to drive a reaction exceeds about 1.23 volts referenced against the standard hydrogen electrode. Electrolysis of water can be detected via one or more sensors and cease or modulate (increase or decrease) current delivery and/or driving voltage if electrolysis is detected in some cases. The sensors can also detect in some cases whether the silver-silver chloride reaction is exclusively, substantially exclusively, or predominantly occurring rather than electrolysis, hydrolysis, or a redox water reaction, for example. Furthermore, the amount of charge that can be delivered by such a system is not limited by surface area reactions such as in the case of platinum electrodes which form a monolayer of platinum-hydride on the electrode surface before the available platinum for reaction is exhausted leading to other potentially harmful products forming if the reaction is continued to be driven.

In contrast, in an aqueous environment when silver-chloride is reduced it forms solid silver and releases a chloride ion into solution and vice-versa. The reaction in each direction is only limited by the quantity of reactant available so the reaction is in effect limited by the total volume of reactant available compared to being limited to surface area. As such, the reaction can utilize an amount of reactant greater than that of the initial, unreacted surface area of the electrode, such as about or at least about 110%, 120%, 130%, 140%, 150%, 160%, 170%, 180%, 190%, 200%, 250%, 300%, 500%, 1,000%, 5,000%, 10,000%, 25,000%, 50,000%, 100,000%, 500,000%, 1,000,000%, 2,500,000%, 5,000,000%, 10,000,000%, 25,000,000%, 50,000,000%, 100,000,000%, 250,000,000%, 500,000,000%, 1,000,000,000%, or more of the initial total, unreacted surface area of the electrode, or ranges including any two of the aforementioned values and depending on the volume of silver utilized. Therefore, substantially more, and in some cases orders of magnitude more charge can be advantageously delivered to body tissue while remaining below the electrolysis threshold.

For example, a platinum or platinum-iridium electrode might deliver 5 microcoulombs per pulse in a 5 mA pulse for 1 millisecond. With embodiment as disclosed herein, it can be possible to achieve about or at least about 1,000×, 5,000×, 10,000×, 50,000×, 100,000×, or more times this charge using DC delivery in the form of a 5 mA pulse with 10 second duration. This may be achieved, for example, by creating a 1 micron coating of AgCl on an electrode of nominal geometry of 3.5 mm length (or between about 1 mm and about 10 mm in length, between about 1 mm and about 5 mm in length, or between about 3 mm and about 4 mm in length) and 1.4 mm diameter (or between about 0.5 mm and about 5 mm in diameter, between about 0.5 mm and about 3 mm in diameter, or between about 1 mm and about 2 mm in diameter) comparable to existing platinum electrodes. One skilled in the art will appreciate that depending on configuration and reservoir of silver-chloride available, the amount of charge delivered can increase to 10,000×, 100,000×, 1,000,000×, 10,000,000×, 100,000,000× or more, or ranges incorporating any two of the aforementioned values, compared to that achievable using a conventional platinum electrode. The silver-silver chloride complex can thus be uniquely situated for use in body environments because the reaction chemistry involves chloride ions which are one of the most readily available ions in and around body tissue.

FIG. 5C shows how current being delivered to the electrode cell (EICCC) (top) can be associated with charge delivered to the electrode cell-nerve interface (middle) to provide nerve block (bottom) while enabling zero net charge transfer with long charge phases. In Phase 1, charge can be delivered to the electrode cell with a given polarity and constant or variable ramp rate (R1) to then provide a constant or variable current (C1) with subsequent constant or variable ramp (R2) back to zero current. Phase 1 may be, for example, of duration up to 1 second, 1 minute, 1 hour, 1 day, 1 month, or 1 year, or longer. In some embodiments, Phase 1 average current is non-zero, but instantaneous current may at times be zero. In some embodiments, Phase 1 is either cathodic or anodic but not both. The initial phase, Phase 1, may be followed by an interphase interval (Interval 1) between cathodic and anodic phases that is greater than or equal to zero seconds. Subsequent to this interval, a second current delivery phase (Phase 2) which may be of duration up to 1 second, 1 minute, 1 hour, 1 day, 1 month, or 1 year, or longer can be applied. This second phase is of opposite polarity to Phase 1 where average current is non-zero, but instantaneous current may at times be zero. In Phase 2, charge can be delivered to the electrode cell with a given polarity and constant or variable ramp rate (R3) to then provide a constant or variable current (C2) with subsequent constant or variable ramp (R4) back to zero current. This Phase 2 may then be followed by another interphase interval (Interval 2) that is greater than or equal to zero seconds.

The waveform in FIG. 5C may be repeated with identical or differing amplitude and duration parameters as a previous waveform whereby the waveform may be programmed or adjusted by a clinician and/or patient and/or caregiver and/or control system. Adjustments may include adjusting the currents in Phase 1 and Phase 2 such that currents are ramped up and/or down as well as adjusting the durations of the phases t1-t0 and t3-t2, respectively. Interphase intervals can also be adjusted such that their durations t2-t1 and t4-t3 are lengthened or shortened. Any delivery or interphase period could be, for example, at least about, about, or no more than about 1, 5, 10, 15, 20, 25, 30, 35, 40, 45, 50, or 55 seconds; 1, 2, 3, 4, 5, 6, 7, 8, 9, 10, 15, 30, 60, 120 minutes; 3, 4, 5, 6, 7, 8, 9, 10, 12, 16, 18, or 24 hours; 2, 3, 4, 5, 6, 7, 14, 21, 28, 30, 45, 60, 75, 90, or more days; 4, 5, 6, 7, 8, 9, 10, 11, 12, 18, 24 or more months; or ranges including any two of the aforementioned values.

At the nerve interface the current delivered to the electrode cell can generate an increase in charge (positive or negative) delivered to the nerve interface as shown and may be linear as shown or generally increasing in a linear or non-linear fashion in Phase 1. The net charge delivered remains roughly constant during the gap phase or Interval 1 then returns to zero during Phase 2. Initially, the period of nerve inhibition (FIG. 5C bottom) is initiated somewhere during the beginning of Phase 1 and nerve block will remain active (solid line) while charge is being delivered to the nerve interface. However, nerve suppression as defined by continued nerve block after removal of current delivery to the electrode cell may continue after current delivery to the nerve is stopped and may persist into the Interval 1 period (ii), extend into Phase 2 (iii) independent of the opposite polarity current being delivered, or into Interval 2 (iv), or beyond (not pictured). Tuning of these parameters can lead to the placement of the nerve into a state of hypersuppression as seen in FIG. 5C (ii), (iii), (iv) in which nerve suppression may occur for durations greater than one minute after removal of DC delivery.

In FIG. 5D waveform patterns delivered to an electrode cell to facilitate nerve inhibition are shown in which current delivered to the electrode cell is shown along with corresponding nerve block periods including hypersuppression regions in which nerve block occurs regardless of current being delivered to the nerve tissue. The electrode cell system is designed such that the cathode and/or anode phase is designed to place the neural tissue in a state of not being conductive (or partially conductive) after the cessation of the current for periods longer than one minute (hypersuppression) after current delivery. In FIG. 5D(i) an electron current has been delivered to the electrode cell to generate an ion current at the neural tissue facilitating a nerve block. After this initial current delivery, the neural tissue then enters a state of hypersuppression whereby in the absence of additional current delivery to the neural tissue, the nerve cannot conduct signals or is not fully conductive.

The neural tissue suppressed state may also be extended as shown in FIG. 5D(ii) in which a secondary current is delivered to the neural tissue a period of time after the initial current has been delivered where the delivery of the extension current may occur up to one minute after the initial current is delivered or after periods of longer than one minute (hypersuppression). This pattern of suppression extension may be repeated for a defined period of time or indefinitely with constant or variable length intervals between current delivery phases.

To maintain the electrode cell in a charge neutral state over repeated uses, current with opposite polarity may be applied after the initial nerve block current is applied and has placed the nerve into a state of hypersuppression (FIG. 5D(iii)). Subsequently, current of the original polarity can be applied to induce additional hypersuppression extension as shown. In this manner the nerve tissue can be repeatedly “dosed” with anodic and/or cathodic safe DC current to maintain the neural tissue in a state of hypersuppression. The hypersuppression duration is longer in duration than a cathodic and/or anodic delivery phase, allowing for complete net charge reversal during hypersuppression. In these examples, the duration and/or amplitude of the cathode and/or anode phase(s) can be programmed to influence the duration and completeness of the nerve block after current delivery has stopped.

FIG. 5E shows an embodiment of an electron-ion current conversion cell (EICCC) 100 which is connected via an electrically insulated lead 112 to a current source 114. The EICCC 100 comprises a traditional electrode (electrode) 104 material (e.g., metal, carbon, etc.) connected to an ionically conductive material (ion conductor 106) which then interfaces with the nerve tissue N, or tissue in proximity to nerve tissue. In some embodiments, the interface can be within about 3 cm, 2.5 cm, 2 cm, 1.5 cm, 1 cm, 5 mm, 2 mm, 1 mm, 0.5 mm, 0.1 mm, or less in proximity to nerve tissue. One skilled in the art will appreciate that the conventional electrode 104 and ionically conductive material 106 may be attached in a multitude of ways such as shown abutting or in an interlocking fashion and the like. The electrode 104 may be inserted within the ion conductor 106 or vice-versa.

As shown in FIG. 5F, in one configuration when the electron current is of one polarity as designated by the positive axis, the nerve block is shown to be active and when the polarity of the current is reversed as designated by the negative axis, the nerve block is shown to be inactive. It should be appreciated that in the case in which the initial blocking current is applied to the nerve N in such a manner to induce a state of hypersuppression, the nerve may remain N blocked during the current reversal period. During the current reversal period, the EICCC 100 is in a resetting phase in which the reaction used to generate the ion current is reversed to bring the EICCC 100 components back towards their original state for subsequent blocking current generation.

FIG. 5G shows a similar configuration to FIG. 5E but with sequestration screens 118, 120 that respectively separate the traditional electrode 104 from the ion conductor 106 and the ion conductor 106 from the nerve N itself, or nerve adjacent tissue. One of ordinary skill in the art will appreciate that one, two, or more or no screens may be used, or any combination thereof. The screens 118, 120 are configured to selectively allow certain ions to transfer between the respective materials while restricting the movement of other ions whose movement is not desired, for example to maintain reaction species such as Cl− near the electrode. The screens 118, 120 may be comprised of an ionically selective membrane such as an anion exchange membrane that only allows anions to pass through it. FIG. 5H illustrates current vs. time and nerve block status vs. time charts similar to FIG. 5F.

FIG. 5I shows a similar configuration to FIG. 5G but also includes a feedback sensor 122 that monitors the state of the nerve tissue N and/or region proximal to the nerve N. In some embodiments a sensor 122 may be located proximal or distal to the electrode-nerve interface in order to enable measurement of local compound action potential to provide feedback to the current source 114 and EICCC 210 to enable it to modulate electrode current and nerve interface electrode potential to maintain hypersuppression. In some embodiments the sensor 122 may measure nerve tissue voltage signals and use that information as feedback to modulate the current and electric potential generated at the nerve interface. In some embodiments the electric potential is modulated such that the nerve cells are maintained in a depolarization state in which action potentials cannot propagate along the nerve cells. In some embodiments the sensor 122 comprises a reference electrode whereby the potential difference between one or more working electrodes 104 and the reference electrode can be monitored and used as feedback to the current source 114 to ensure proper operating range of the EICCC 210. The implantable packaging may contain an integrated reference or counter electrode. FIG. 5J illustrates current vs. time and nerve block status vs. time charts similar to FIG. 5H.

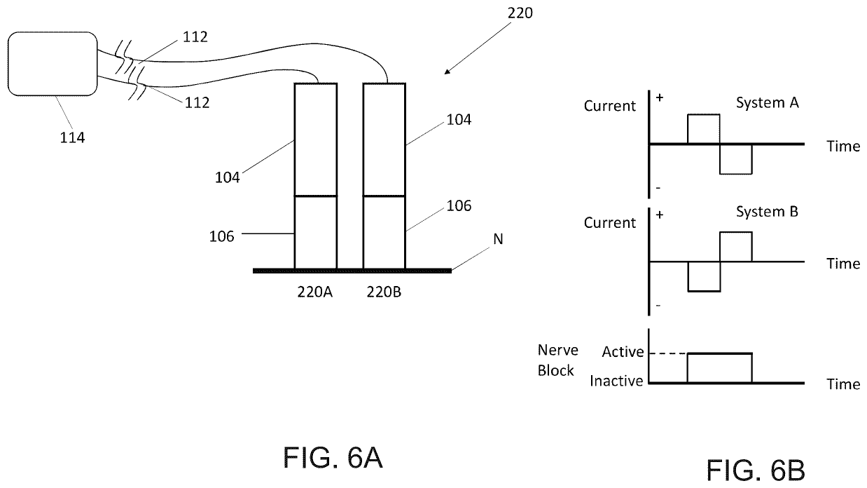

FIG. 6A shows a dual electrode system in which two EICCCs 220A, 220B interface with a nerve N or nerve adjacent tissue. The system of FIG. 6A is also suitable for performing any of the neural inhibiting methods, including the cardiac neural inhibiting methods, described above. The two electrodes 220A, 220B are driven with currents of opposite polarities as a function of time such that when one is in an active blocking phase, the other is in an inactive non-blocking phase which resets the electrode for blocking once the current polarity is again reversed as shown in FIG. 6B. With this configuration a constant block can be maintained along the nerve N. It should be appreciated that in the case in which a blocking current is applied to the nerve N in such a manner to induce a state of hypersuppression, the nerve block may remain active during the current reversal period of the electrodes 220A, 220B. One skilled in the art will appreciate that the driving currents for the two electrodes 220A, 220B may be spaced apart in time during which no current is driving one or both electrodes 220A, 220B and that during this period block may be maintained if the nerve N is in a state of suppression. Similarly, the driving currents may be of different durations dependent on the electrodes 220A, 220B themselves and any recovery time of the nerve N during which signal remains blocked while no blocking current is being applied. The electrodes 220A, 220B may be oriented as shown in series axially along a nerve N or oriented on opposite sides to the nerve tissue itself.

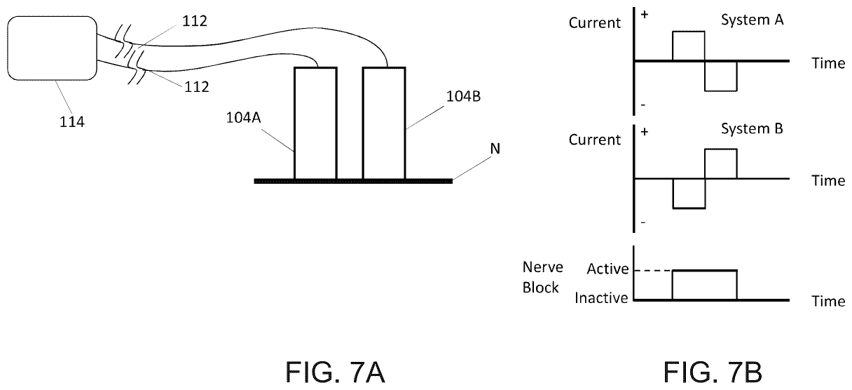

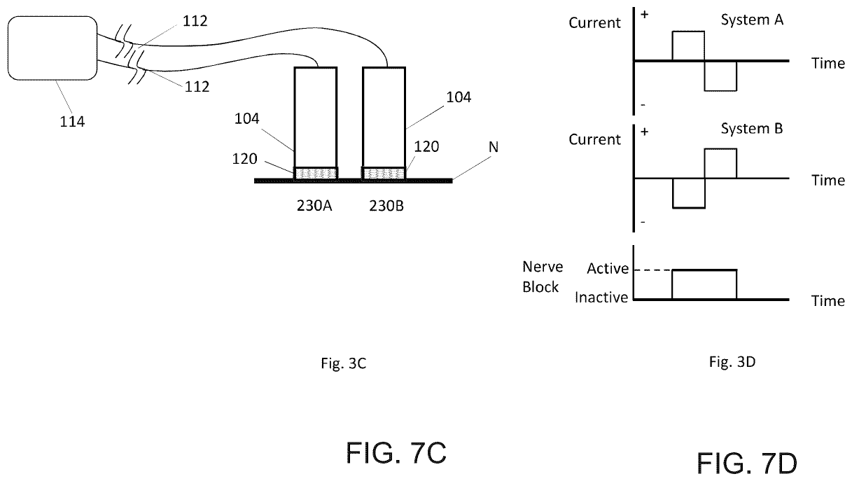

FIGS. 7A-B show an embodiment where dual traditional electrodes 104A, 104B interface with a nerve N but are driven from a current source via electrically insulated leads 112 with currents of opposite polarities such that when one is in a blocking phase, the other is in a non-blocking phase which resets the electrode for blocking once the current polarity is again reversed. With this configuration a constant block can be maintained along the nerve. The electrodes 104A, 104B may be oriented as shown in series along a nerve N or oriented on opposite sides to the nerve tissue itself.

FIGS. 7C-D show an embodiment where dual EICCCs 230A, 230B interface with a nerve N but are driven with currents of opposite polarities such that when one is in a blocking phase, the other is in a non-blocking phase which resets the electrode for blocking once the current polarity is again reversed. With this configuration a constant block can be maintained along the nerve N as illustrated in FIG. 7D. The electrodes 230A, 230B interface with the nerve N via screens that sequester deleterious byproducts from dangerous electrochemical reactions to protect the nerve N. The screens may include an ionically selective membrane such as an anion exchange membrane that only allows, for example, anions to pass through it (but not cations).

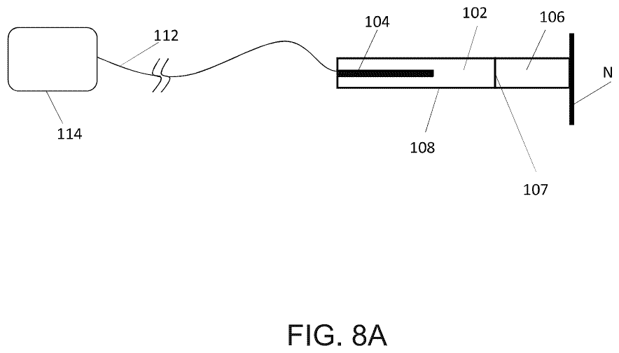

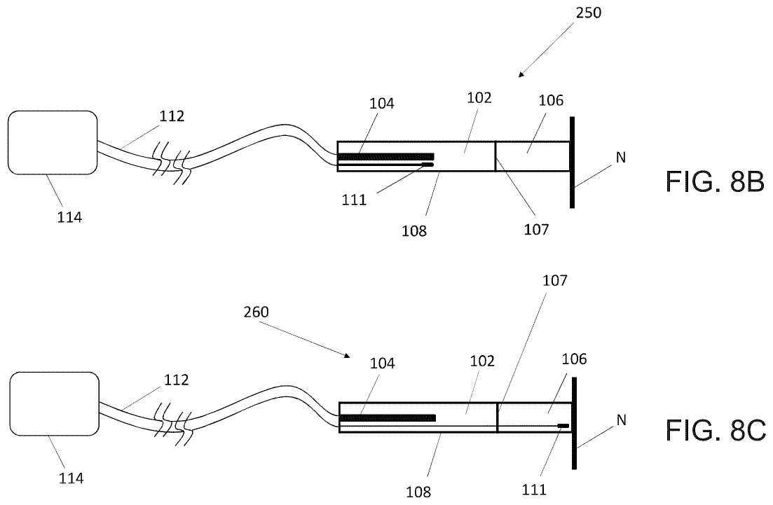

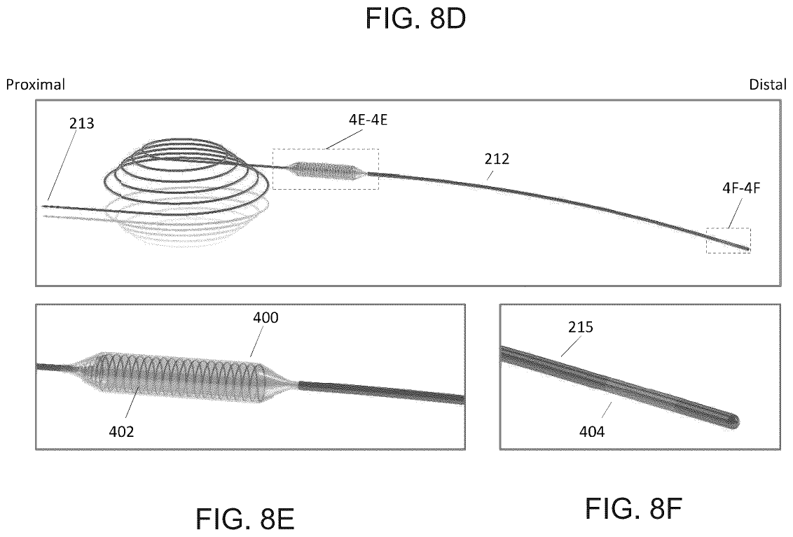

FIG. 8A shows an embodiment of an EICCC electrode 240 in which an electrode 104 is immersed in an electrolyte solution 102 which fluidly in is contact with an ion-conductive material 106 such as a hydrogel, gel or other polymer that electrically contacts the nerve tissue N or area proximal to the nerve tissue. The EICCC 240 electrode also comprises an electrically insulated enclosure 108 housing the traditional electrode 104, electrolyte 102, ion conducting material 106 with an aperture (near 110) to enable electrical contact with the nerve or area proximal to the nerve tissue. The electrolyte-hydrogel 107 interface can alternatively be mediated by an ion selective screen or an ion conductive polymer to sequester by products of any electrochemical reactions to the aqueous region of the cell. The system further optionally comprises a current delivery lead 112 between the current source 114 and the electrode 104. The current source 114 may be located external or internal to the body depending on the application need. An exemplary embodiment of the EICCC 240 comprises a silver, silver-chloride (Ag/AgCl) electrode in a 0.9% saline solution in fluid contact with an electrolyte saturated hydrogel (agar preparation with 0.9% saline). In other examples, the electrode material may comprise metal, carbon, conductive polymers materials and may be configured in a high surface area to volume configuration that may include configurations such as open-celled foam configurations, sintered particle configurations, dendritic configurations or the like.

FIG. 8B shows a system 250 similar to that shown in FIG. 8A with the addition of a reference electrode 111 in proximity to the electrode (working electrode) 104 to monitor voltage drop across the working electrode 104 for EICCC monitoring purposes. For example, to ensure that the electrode 104 is being driven under the desired conditions to ensure that the proper electrochemical reactions are occurring.

FIG. 8C shows a system 260 similar to that shown in FIG. 8A with the addition of a reference electrode 111 in proximity to the nerve tissue interface to monitor voltage drop across the EICCC 260 to the nerve tissue for EICCC monitoring purposes. For example, to ensure that the electrode 104 is being driven under the desired conditions to ensure that the proper electrochemical reactions are occurring.

FIGS. 8D-F show an embodiment of an electrode lead 212 configured to plug into and extend from a current source (not shown, near end 213) that might take the form of conventional IPGs (implantable pulse generators). FIG. 8F is a close-up view of 4E-4E of FIG. 8D. FIG. 8F is a close-up view of 4F-4F in FIG. 8D. This configuration could be similar to as that shown in FIG. 8A except that the nerve interface hydrogel shown in FIG. 8A is removed and the nerve tissue interface comprises a screen or porous frit 404 which contains the electrolyte solution but allows ions to pass to the nerve tissue environment. A connector 213 to the current source is shown with an electrically conductive portion of the electrode lead 213 that extends distal from the connector 213 to the EICCC 400 which couples the electron current to an ionic current via electrochemical reactions. The coiled electrode 402 converts electrical current to ionic current in the EICCC 400 which is then transmitted toward the distal portion of the lead which can be positioned in proximity to the target nerve tissue. Contact with the nerve tissue environment occurs via the ionic current which exits the screen/porous frit 404 that can manipulate the nerve environment.

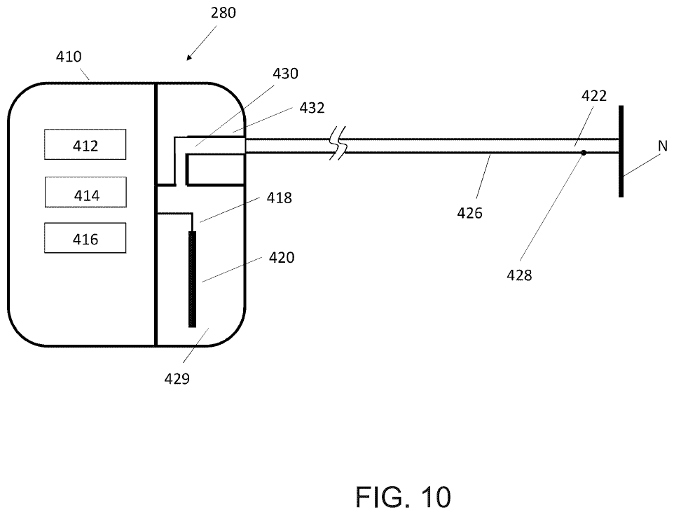

FIG. 10 shows a schematic embodiment of an EICCC integrated within a hermitically sealed enclosure 410 which contains the current source 412, battery 414 or power supply, and controller 416 to drive the EICCC 280. The EICCC 280 is directly connected to the current source 412 as illustrated and comprises a lead 418 from the current source 412 and an electrode 420 immersed in an electrolyte solution 429 which fluidly is in contact with an ion conductive material 422 such as a hydrogel which in turn contacts the nerve tissue N to be blocked. In this embodiment the electrode element 420 of the EICCC 280 is located relatively proximal to the current source 412 while the nerve contacting lead is located relatively more distally from the current source 412 and extends to the nerve location N. Also illustrated is the ion conductive conduit (e.g., hydrogel connector) 430, connector elementals 432, insulated enclosure 420, and ion conducting electrode lead 428.

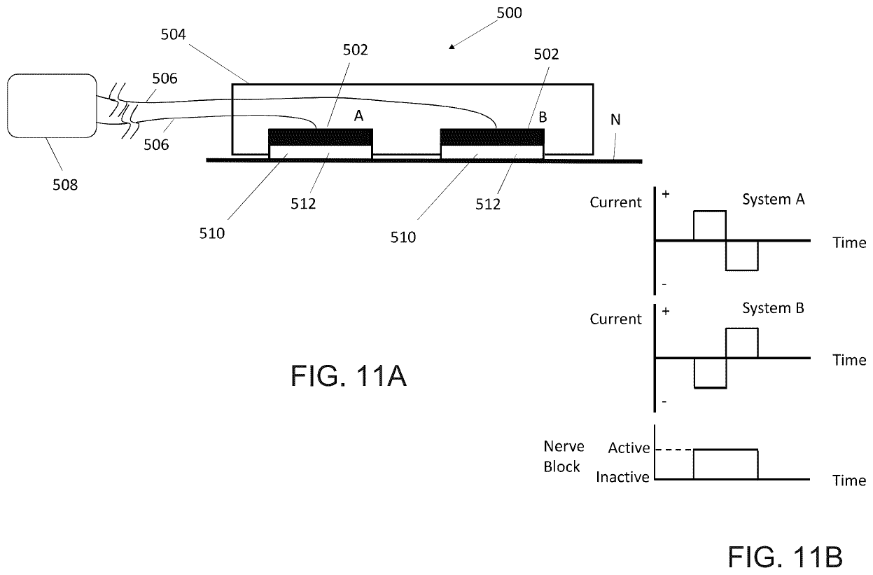

FIGS. 11A-B show an embodiment of an electrode configuration 500 in which two electrode contacts are housed within the same electrically insulated enclosure 504. The electrodes 502A, 502B are in ionic contact with ion conducting materials 510/pads 512 that interface with the nerve tissue N and/or area proximal to the nerve. Each electrode 502A, 502B is in electrical communication via its own conductive lead 506 that is driven by the current source 508. The internal electrodes 502A, 502B can be driven cyclically with opposite current polarities to provide a constant nerve block. The current source 508 may be configured to be implantable within the body such that any leads 506 and electrodes 502A, 502B are also fully contained within the body. Alternatively, the current source 508 may be configured to remain outside the body and can be connected via wired or wireless connections in this or other embodiments.

Waveforms

Nerve inhibiting therapy, including cardiac nerve inhibiting therapy may utilize a variety of different electrical waveforms to inhibit nerve activity during therapy. For example, in some embodiments, an electrical therapy system may utilize any of ultra-low frequency waveform, high-frequency alternating current, high-frequency alternating current combined with a DC component to mitigate onset response, monophasic direct current, charge-balanced direct current carousel (CBDCC), charge-balanced direct current, biphasic direct current, complex ultra-low frequency waveform, low/moderate frequency waveform for generating excitation-induced information lesion in the target tissue.

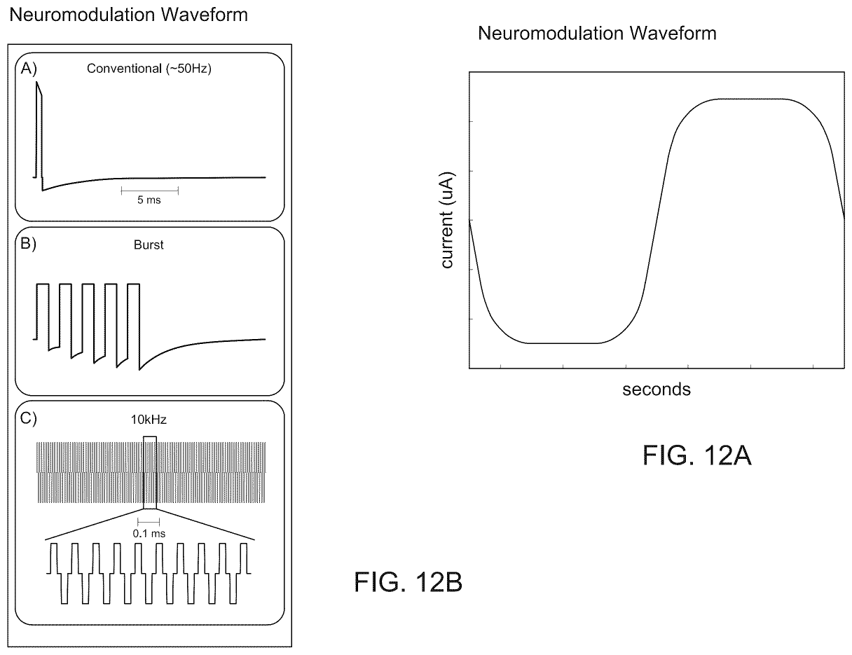

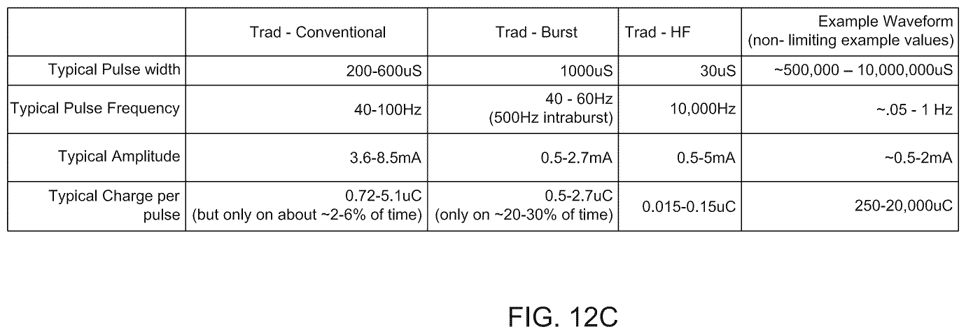

FIG. 12A illustrates example neural inhibiting waveforms, including conventional, burst, and high frequency (e.g., greater than about 1 kHz, or more). As illustrated, each of the foregoing waveforms can include sharp angles or corners. In contrast, neural inhibiting waveform illustrated in FIG. 12B includes curves in place of sharp angles, corners, or edges, which can provide any number of the benefits explained herein. The waveform illustrated in FIG. 12B can be referred to as a spline waveform shape and/or smooth waveform shape instead of the square wave or sharp corner shapes illustrated in FIG. 12A.

FIG. 12C illustrates a table with example non-limiting pulse widths, pulse frequencies, amplitudes, and charge per pulse values for each of the conventional, burst, high frequency, and curved neural inhibiting waveforms. As provided in the table, the charge per pulse value for the curved waveform illustrated in FIG. 12B can be much greater than, and even orders of magnitude greater than that of the other waveforms.

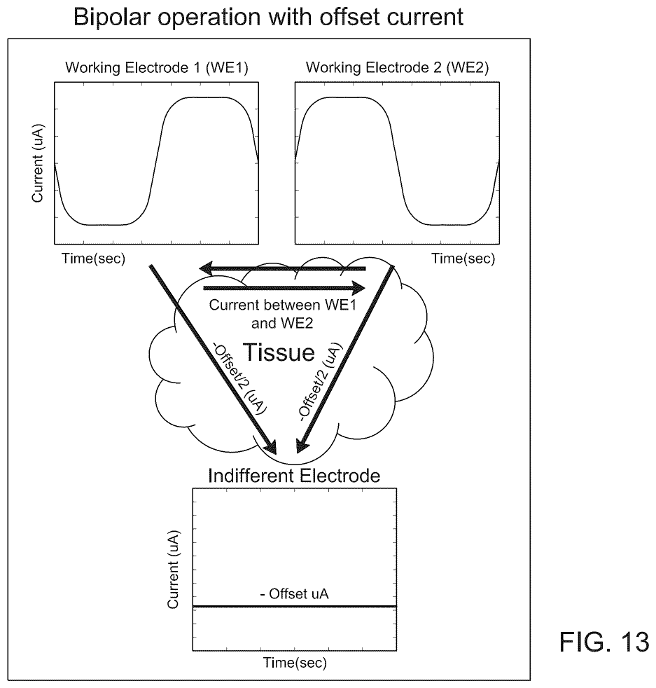

FIG. 13 illustrates an example bipolar operation with bias (offset) current. In a bipolar configuration, balanced low frequency stimulation current is conducted between two working electrodes (WE1, WE2). An offset current is added to each WE electrode and is absorbed by the indifferent electrode, which can be either a surface or implanted transcutaneous electrode that can accommodate the opposite voltage of the working electrode. FIG. 13 shows a non-limiting example of the bipolar arrangement with even bias (offset) currents for each electrode and a constant DC bias (offset) current. In a monopolar arrangement, stimulation current is conducted between the working electrode (WE) and a counter or indifferent electrode (IE). The counter or indifferent electrode can be a surface or implanted electrode or transcutaneous electrode that can accommodate the opposite voltage of the working electrode. Stimulation current can include an ultra-low frequency AC component and an offset current can be added such that the WE is biased in the correct voltage range.

Pulse Generators

In some embodiments, the electrical therapy system may utilize a pulse generator in the delivery of the nerve inhibiting therapy, including cardiac nerve inhibiting therapy. In some embodiments, the electrical therapy system may utilize an external or implantable pulse generator, such as an implantable pulse generator placed in a surgical pocket, an implantable pulse generator that does not require a surgical pocket (e.g., is placed near the target nerve structure in same procedure), or an implantable pulse generator that is contained within the therapy leads.

In some embodiments, the pulse generator may be placed externally. For example, in some embodiments, the pulse generator may be affixed to the skin (e.g., via an adhesive, suture, wrapping), worn on the patient (e.g., in a belt, backpack, pouch, pocket, etc.), or handheld and connects wirelessly to the implanted leads.

In some embodiments, the pulse generator may be external capital equipment. For example, in some embodiments, the pulse generator may be external and separate from the patient and connects to the implanted leads (e.g., a piece of capital equipment). Further, the pulse generator may be located in the ICU, in a Catheter Lab, in an inpatient setting, and/or in in a remote telemetry unit.

In some embodiments, the pulse generator may be powered via a battery. For example, the pulse generator may be powered via: primary cell battery (e.g., does not require recharging), replaceable primary cell battery, rechargeable battery. In some embodiments, the pulse generator may be powered via wireless charging (e.g., inductive, ultrasound, optical, magnetic, removable rechargeable battery). In some embodiments, the pulse generator may be battery-free and may be powered via transcutaneous energy delivery (e.g., inductive, ultrasound, optical, magnetic). In some embodiments, the pulse generator may be powered via energy harvesting from the body or AC power from the building.

Leads

In some embodiments, delivery of nerve inhibiting therapy, including cardiac nerve inhibiting therapy may be accomplished in various ways. For example, in some embodiments of an electrical therapy system, therapy may be delivered via electrical leads. The electrical lead may include both electrical and non-electrical electrodes. For example, in some embodiments, electrical electrodes of the electrical therapy system may include a percutaneous system cylindrical lead, a paddle lead, a cuff electrode, a pellet electrode, a pin electrode, skin-mounted electrode (e.g., 3M Red Dot), a catheter style electrode (e.g., SINE electrode). In another example, in some embodiments, non-electrical electrodes of the electrical therapy system may include ultrasound (e.g., piezoelectric transducer), pharmaceutical (e.g., drug-eluting catheter, continuous or repeated drip infusion catheter, thermal transducers).

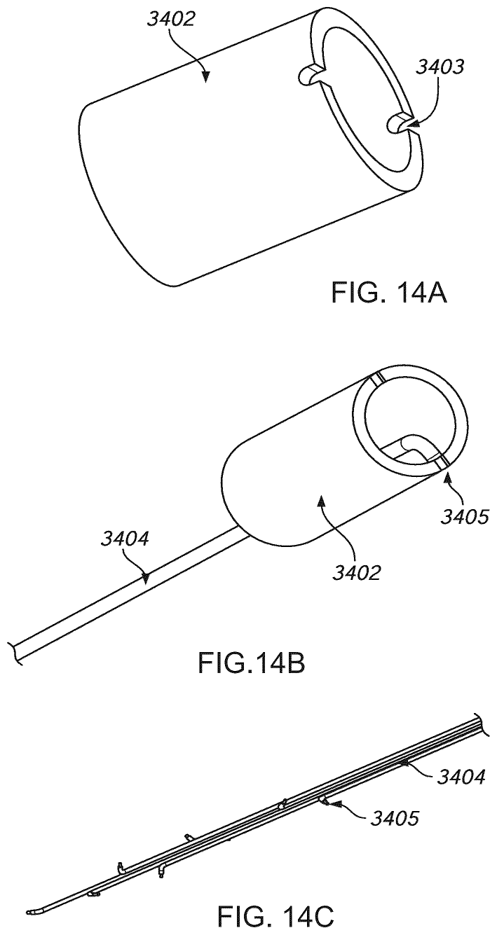

FIGS. 14A-14C illustrate one embodiment of a lead having a generally tubular, e.g., cylindrical, lead contact 3402 that can include a sidewall with optional distal notches 3403 and an interior lumen. One, two, or more conduits 3404 can be housed at least partially within each lead contact 3402 with conductive end portions 3405 in electrical communication with the lead contact 3402. As illustrated in FIG. 14B, the conductive end portion 3405 can be positioned, which can include retained, within the distal notch 3403. As shown in FIG. 14C, a plurality of conduits 3404 can be grouped together with each having a conduct end portion 3405 extending outward.

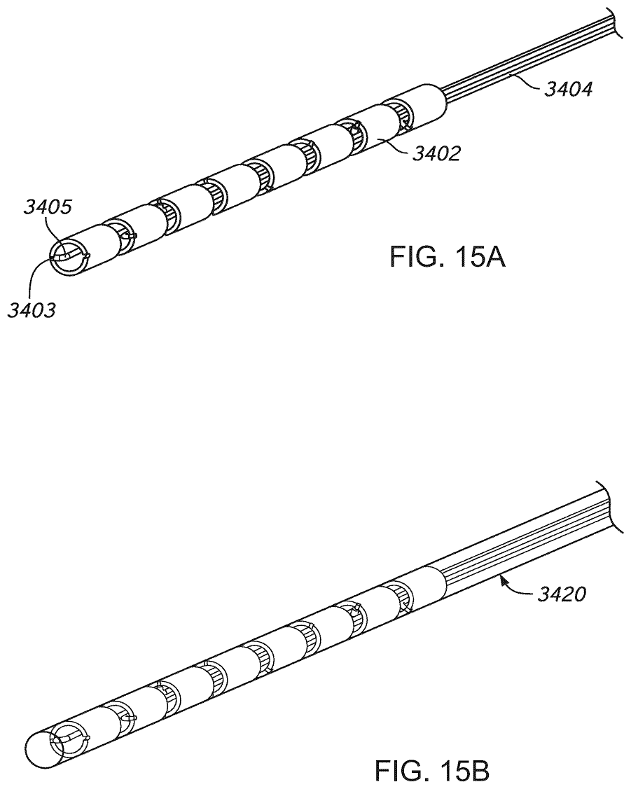

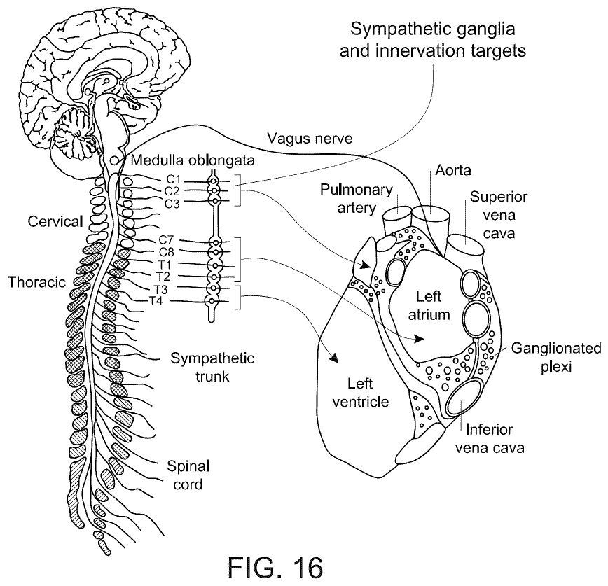

FIGS. 15A and 15B illustrate additional lead embodiments, illustrating conduits and spaced-apart lead contacts as previously described. FIG. 15A illustrates contacts 3402 without binding material or a coating layer, while FIG. 15B illustrates contacts connected with a binding material 3420 which may, for example, comprise silicone that has been overmolded onto and throughout the other components except where excluded such as along the outer electrode contact surfaces. Optionally a coating layer 3422 may be situated as a surface layer over the lead embodiment. In some embodiments this coating layer may comprise an ionically conductive polymer such as an anion exchange membrane.

Nerve Targets

In some embodiments, nerve inhibiting therapy, including cardiac nerve inhibiting therapy, may target one or more nerves to achieve the intended therapeutic effect. For example, alternative nerve targets may include: the paravertebral sympathetic chain, such as the stellate ganglia, thoracic levels (T1-T4, T1-T2). In some embodiments, alternative nerve targets may also include the ansa subclavia, the cardiac plexus, the spinal cord (including cervical region C6-C7), a combination of thoracic and cervical regions, the dorsal root ganglion. In some embodiments, for all preceding nerve targets, cardiac nerve inhibiting therapy may be delivered unilaterally, bilaterally, or even a combination of one or more targets or regions.

In addition, the current source may be located outside the body of the patient permanently or temporarily to enable nerve inhibition. The electrodes may also be removed once deemed unnecessary thus provided a temporary nerve inhibition as desired. The nerve inhibition may also be turned on and off periodically by modulating the current source as required to enable sensation during procedures that require patient feedback for example.

Nerve inhibition at specific dermatomes can be used to localize therapeutic pain reduction due to neuralgias, angina, ischemic pain, and complex regional pain syndrome (CRPS). In the case of angina, cervical spinal level nerve roots C6 and C7 have been implicated as frequently involved with the associated pain, and localized inhibition at one or both of these levels (with or without inhibition of additional nerves at other levels) could be used to help manage this pain. For example, complex regional pain syndrome (CRPS) is often localized to a single limb and generating a localized block can provide more specific pain block for the source of pain. For example, the lumbar dorsal root ganglia at levels L2, L3, L4 have been shown to be able to reduce knee pain on the ipsilateral side of the spine using conventional stimulation techniques. Ischemic pain frequently is localized particularly for patients with poor extremity circulation and may be similarly mitigated by targeting the appropriate nerves for inhibition.

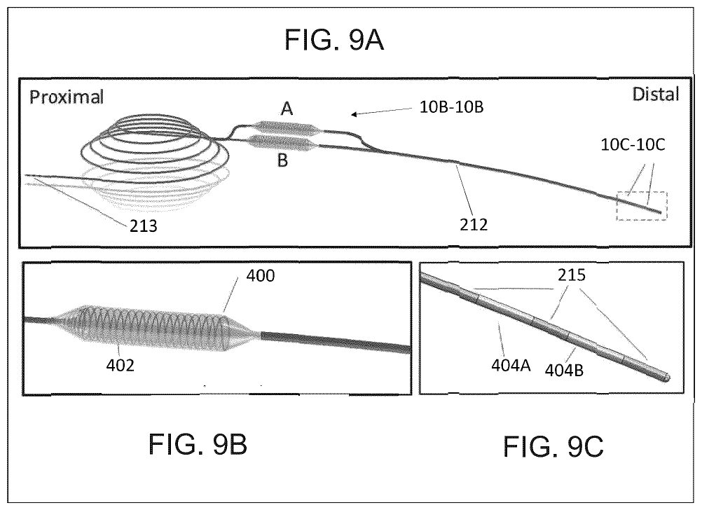

In some embodiments a multiple electrode leads such as illustrated in FIG. 15B and FIG. 9A, for example, may be positioned to target a desired nerve. Furthermore, the electrodes may be configured to have a multitude of tissue contacting regions whose outputs can be individually adjusted to optimize the nerve inhibition. An embodiment of an EICCC electrode is shown in FIGS. 9A-9C in which multiple tissue interfaces 404A, 404B are present on the electrode 402 and are individually addressable. FIG. 9B is a close-up view of 10B-10B of FIG. 9A. FIG. 9C is a close-up view of 10C-10C of FIG. 9A. In this embodiment, a dual system of EICCCs 400 are present and have parallel lumens that are individually associated with each nerve tissue interface region. In FIG. 9A the nerve tissue interface region and associated EICCC are designated by matching letter labels, in this case A and B. Adjusting the current input and corresponding output at the distal end of the electrode can enable electric field shaping to facilitate desired nerve block while minimizing block of undesired structures. Alternative embodiments are illustrated in previously discussed figures. In such embodiments, individual electrodes are also individually addressable and can be tuned to enable desired nerve inhibition.

Using these methods of placement of nerve inhibiting electrodes, specific targets for nerve inhibition can be achieved. For example, trunk pain which is moderated by the thoracic vertebral levels can be modulated by placing leads along the thoracic spine while neck pain may be moderated by providing neural inhibition and/or suppression in the cervical spine. Upper limb pain may be moderated by providing a combination of cervical and thoracic level neural inhibition and/or suppression while lower limb pain may be moderated by a combination of lumbar and sacral level neural inhibition and/or suppression in the spine.

Generation of pain inhibition can be used to facilitate peri-procedural pain block where motor control and non-pain sensations are desired. For example, in labor and delivery of a child, one of the challenges with pain management particularly with epidural anesthesia is the reduction in ability to be sensate in the lower body. Due to the non-specific nature of the delivered anesthesia in the epidural space sensory, pain, and motor neurons are impacted. The epidural anesthesia can lead to difficulty with generating pushing force during the birthing process and can lead to numbness a few hours after birth impairing motor abilities such as the ability to walk. In some instances, epidurals are further implicated in fetal and newborn health including breastfeeding difficulty. Using the neural inhibiting electrodes described above to target the spinothalamic tract and/or dorsal root ganglia, the undesired pain can be targeted without generating the side effects (or reducing side effects) associated with current epidural anesthesia techniques because only the pain tracts are targeted and not any other motor or sensory tracts. Furthermore, in the case in which ionic current is delivered to the nerve tissue in a reversible blocking fashion, the stopping of block can enable the patient to immediately be restored to normal pain sensation if desired and any off-target block can be reversed enabling immediate body function restoration.

Beyond central nervous system interventions, a safe direct current inhibition can also be facilitated in the peripheral nervous system in which EICCC electrodes are placed in contact or in proximity to peripheral nerves to facilitate nerve inhibition. Specific pain targets include focal pain, phantom limb pain, neuroma pain, and neuralgias. Targeting the peripheral nerves proximally (i.e., closer to the spinal cord) from the site of pain for neural inhibition can suppress pain from the distal site. Specific to neuralgias, postherpetic neuralgia (after shingles) can be targeted based on the presentation of the outbreak which will trace specific dermatomes. For trigeminal neuralgia, the trigeminal nerve (and/or trigeminal ganglion and/or trigeminal nucleus in the brainstem) can be targeted for inhibition to reduce pain that commonly manifests as facial pain. Another target is the glossopharyngeal nerve which produces pain in the neck and throat. Neuralgia in extremities such as the hands, arms, feet, and legs as frequently caused due to diabetes-related neuropathies are also potential targets.

Outside of pain reduction, nerve inhibition and activity suppression can be used to improve cardiovascular health in specific targeted ways. Hypertension which is implicated as a leading cause of cardiovascular disease has been found to be able to be moderated by modulation of the renal nerves to reduce activation of the sympathetic nervous system. Current techniques exist to denervate or ablate these nerves using a variety of energy sources such as ultrasound and radiofrequency energy. Using the tools described herein, selective nerve block can be used to facilitate activity reduction in the renal nerves and sympathetic nervous systems to facilitate reduction in hypertension. The delivered nerve inhibiting current can also be adjusted to fit the individual physiological response to sympathetic nerve block which cannot be done currently with destructive methods such as ablation.

Heart failure is another target disease state with known association with upregulation of the sympathetic nervous system. By using a nerve inhibiting electrode to moderate the sympathetic ganglia, particularly reducing activity of the cervical sympathetic ganglia, excessive heart activity can be reduced to mitigate overworking of the heart. Similar to dorsal root ganglion access, the cervical ganglia may be accessed for inhibition. As shown in FIG. 16, the relevant sympathetic ganglia including the cervical and stellate (cervicothoracic) ganglia are shown along with their innervation targets in the heart. Methods of access include posterior access as well as through the pleural cavity.

The systems and methods described in the figures above may be used to generate DC nerve inhibition. Depending on the specific direct current application of nerve inhibition, nerve suppression, or continued inhibition after removal or stopping of the current may occur, and hypersuppression may result for continued nerve inhibition in excess of one minute after removal of the DC source to delay nerve conduction recovery. The nerve inhibition and suppression may be generated in an intermittent or continuous manner depending on the desired application. Means for continuous nerve inhibition have been described that provide for safe delivery of nerve inhibition via ionic current utilizing multiple electrodes or sequenced electrode contact activation enabling a means to modulate nerve conduction safely without necessitating complex mechanical systems. The system may be fully or partially implantable, or completely non-implantable (e.g., transcutaneous) with all tissue contacting materials biocompatible for tissue contact and implantation compatibility.

Additional Applications and Indications

Several embodiments of the previously described systems relate to non-limiting applications in the post-infarct setting to prevent formation of proarrhythmic cardiac substrate or paravertebral sympathetic chain.

Cardiology may be an alternative application, specifically for prevention and/or termination of cardiac arrythmia in the post-MI setting. Here and in following indications, cardiac arrythmias include but are not limited to ventricular arrythmias, atrial arrhythmias, chronic arrhythmias, and acute/episodic arrythmias. In some embodiments, the systems and methods described may be used for prevention and/or termination of cardiac arrythmia following cardiac surgery. Similarly, in some embodiments, the systems and methods describe may be used for prevention and/or termination of cardiac arrhythmia in patients at risk for cardiac arrhythmia, with or without an infarct, such as in the hospital, ICU, after discharge, or outside of a healthcare facility. In some embodiments, the systems and methods described may be used for applications relating to favorable remodeling of the cardiac substrate, paravertebral sympathetic chain, and/or spinal cord following cardiac injury including but not limited to myocardial infarction, cardiac surgery, traumatic injury, substance abuse, metabolic disorders.

In some embodiments, the systems and methods described may be used for applications relating to stabilization of heart rate variability, reduction of inflammation and/or glial activity of the paravertebral sympathetic chain, cardiomyocytes, or other components of the neurocardiological system, and treatment of unstable angina.

In some embodiments, the systems and methods described may be used for applications relating to prevention of non-arrhythmia induced sudden cardiac death after MI, such as cardiogenic shock, ischemic mitral regurgitation, ventricular septal rupture, LV free wall rupture, and pericarditis.

In some embodiments, the systems and methods described may be used for psychology applications, such as the treatment of post-traumatic stress disorder (PTSD) or treatment of chronic anxiety.

In some embodiments, the systems and methods described may be used in vascular applications, such as for the treatment of hyperhidrosis, treatment of Raynaud's phenomena, treatment of coronary artery atherosclerosis, or treatment of vasoconstrictive disorders of the head/neck.

In some embodiments, the systems and methods described may be used for pain-related applications, such as the treatment of angina, sympathetically driven pain, complex regional pain syndrome, post herpetic neuralgia, facial pain, head and neck pain, upper limb pain, phantom/post-amputation pain, cancer pain, oral pain, or vascular headache.

In some embodiments, the systems and methods described may be used for other miscellaneous applications, such as for treatment of lymphedema (e.g., refractory breast cancer lymphedema) and any therapy for which stellate ganglion blocks, surgical sympathectomy, radiofrequency ablation, or medications to reduce sympathetic tone or pain, including upper limb pain, are used.

Additional Considerations

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions.

Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above.

Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims.

Any methods disclosed herein need not be performed in the order recited. The methods disclosed herein include certain actions taken by a practitioner; however, they can also include any third-party instruction of those actions, either expressly or by implication. For example, actions such as “applying direct current to a nerve” includes “instructing the applying of direct current to a nerve.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1. A method to prevent formation of unfavorable sequelae, comprising:
initiating delivering of a neural inhibiting therapy to a nerve associated with an extracardiac sympathetic nervous system of a patient within 40 days of an onset of symptoms associated with acute coronary syndrome,
wherein the neural inhibiting therapy comprises delivering an electrical therapeutic signal to the nerve sufficient to substantially prevent neural activity of the nerve from occurring;
continuing delivery of the neural inhibiting therapy to the nerve associated with the extracardiac sympathetic nervous system for a desired treatment period; and
discontinuing delivery of the neural inhibiting therapy to the nerve after the desired treatment period and enabling neural activity of the nerve to resume.

2. The method of claim 1, further comprising implanting an electrical contact close to the nerve associated with the extracardiac sympathetic nervous system of the patient.

3. The method of claim 2, wherein implanting comprises implanting the electrical contact under ultrasound or fluoroscopy or other image guidance method.

4. The method of claim 1, wherein delivering therapy to the nerve associated with the extracardiac sympathetic nervous system of the patient comprises delivering therapy to a paravertebral chain at thoracic levels T1-T2, stellate ganglia, spinal cord, ansa subclavia or dorsal root ganglia.

5. The method of claim 1, wherein the acute coronary syndrome comprises an acute myocardial infarction (MI) event, and wherein delivering therapy occurs within 14 days of the MI event, within 2 days of the MI event, before 14 days of the MI event, or during the MI event.

6. The method of claim 1, wherein delivering therapy comprises one or more of delivering a neural inhibiting electrical waveform, delivering an ultra-low frequency waveform, providing a non-electrical therapy, providing thermal energy, providing magnetic energy, or providing ultrasonic energy.

7. The method of claim 1, wherein continuing neural inhibiting comprising inhibiting neural activity for at least 14 days, up to 40 days, until a sympathetic tone is decreased by a desired amount in the absence of the neural inhibiting, until a biomarker level of a biomarker relating to myocardial death drops by a desired amount.

8. The method of claim 7, wherein the biomarker is troponin or any other biomarker described herein.

9. The method of claim 1, wherein deactivating blocking comprises deactivating a waveform generator.

10. The method of claim 1, further comprising removing a treatment device from the patient, wherein the treatment device is configured to perform the method of claim 1.

11. The method of claim 1, wherein removing comprises removing an implant or removing a patch or other device from a skin surface of a patient.

12. A system configured to prevent formation of unfavorable sequalae, comprising:
a current generator;
at least one electrode coupled to the current generator; and
a controller, coupled to the current generator, and configured to control the current generator to perform the method of claim 1.

13. The method of claim 1, wherein the unfavorable sequelae comprise proarrhythmic cardiac and stellate substrates.

14. The method of claim 1, wherein the unfavorable sequelae comprise undesired sympathetic tone in an extracardiac neural circuit.

15. The method of claim 1, wherein the acute coronary syndrome comprises one or more of ST elevated myocardial infarctions (STEMI), non-ST elevated myocardial infarctions (NSTEMI) or unstable angina.

16. A method to reverse formation of unfavorable sequelae, comprising:
initiating delivering of a neural inhibiting therapy to a nerve associated with an extracardiac sympathetic nervous system of a patient 40 days or more after occurrence of a symptom associated with acute coronary syndrome,
wherein the neural inhibiting therapy comprises delivering an electrical therapeutic signal to the nerve sufficient to substantially prevent neural activity of the nerve from occurring;
continuing delivery of the neural inhibiting therapy to the nerve associated with the extracardiac sympathetic nervous system;
assessing a function of cardiac tissue; and
discontinuing delivery of the neural inhibiting therapy to the nerve and enabling neural activity of the nerve to resume in response the assessing.

17. (canceled)

18. (canceled)

19. (canceled)

20. (canceled)

21. The method of claim 16, wherein continuing neural inhibiting comprising inhibiting neural activity for at least 14 days, up to 40 days, until a sympathetic tone is decreased by a desired amount in the absence of the neural inhibiting, until a biomarker level of a biomarker relating to myocardial death drops by a desired amount.

22. The method of claim 21, wherein the biomarker is troponin or any other biomarker described herein.

23. (canceled)

24. (canceled)

25. The method of claim 16, wherein removing comprises removing an implant or removing a patch or other device from a skin surface of a patient.

26. (canceled)

27. (canceled)

28. A method to reduce a sympathetic tone in an extracardiac neural circuit, comprising:
initiating delivering of a neural inhibiting therapy to a nerve associated with an extracardiac sympathetic nervous system of a patient 40 days or more after occurrence of a symptom associated with acute coronary syndrome,
wherein the neural inhibiting therapy comprises delivering an electrical therapeutic signal to the nerve sufficient to substantially prevent neural activity of the nerve from occurring;
continuing delivery of the neural inhibiting therapy to the nerve associated with the extracardiac sympathetic nervous system;
assessing a function of cardiac tissue; and
discontinuing delivery of the neural inhibiting therapy to the nerve after the desired treatment period in response to the assessing and enabling neural activity of the nerve to resume.

29.-38. (canceled)
